(19) United States
(12) Patent Application Publication
(10) Pub. No.: US 2023/0257695 A1
(43) Pub. Date: Aug. 17, 2023

(54) METHODS FOR CREATING EDIBLE SCAFFOLDING USING CHILEAN SEAWEED (COCHAYUYO)

(71) Applicant: Luyef Biotechnologies INC., Wilmington, DE (US)

(72) Inventors: Andrea Villanueva, Santiago (CL); Felipe Sebastian Sanchez, Santiago (CL); Ziomara Gerdtzen, Santiago (CL); Kris Blanchard, Santiago (CL); Pamela Barroso, Santiago (CL); Stephanie Brain, Santiago (CL)

(73) Assignee: Luyef Biotechnologies INC., Wilmington, DE (US)

(21) Appl. No.: 18/072,718

(22) Filed: Dec. 1, 2022

Related U.S. Application Data
(60) Provisional application No. 63/284,659, filed on Dec. 1, 2021.

(51) Int. Cl. C12N 5/00; A23L 17/60; A23L 13/00

(52) U.S. Cl. CPC C12N 5/0068; A23L 17/60; A23L 13/00; C12N 2533/90

(57) ABSTRACT

Methods for creating edible scaffolding using algae, and more specifically, a protocol for the sterilization and decellularization of Durvillaea antarctica, Chilean seaweed, or cochayuyo to prepare the cochayuyo to be suitable for the culture of cells are described herein. The scaffold may be used for cell growth and is composed of sterile decellularized cochayuyo rehydrated with adhesion/carrier proteins. The decellularized algae supports cell growth, differentiation, and nutrient diffusion due to its porosity. The product is completely edible. The methods allow for the seaweed material to be dried and rehydrated without problems before and/or after decellularization. Also, the sample described herein can be easily sterilized without losing its form and can be hydrated with proteins. Additionally, the scaffolding can be used as a microcarrier because it can float inside the cell media, and one wouldn't need to change the cells for differentiation.

The present invention claims priority under 35 USC 119(e) to US Provisional Application No. 63/284,659 filed Dec. 1, 2021, the entire contents of which are incorporated by reference in its entirety.

### FIELD OF THE INVENTION

The field of the invention and its embodiments relate to methods for creating edible scaffolding using algae. More specifically, the field of the invention and its embodiments relate to a protocol for the sterilization and decellularization of Durvillaea antarctica, Chilean seaweed, or cochayuyo to prepare the cochayuyo to be suitable for the culture of cells.

### BACKGROUND OF THE INVENTION

Projections show that a growing population and the demand for meat poses a risk of insufficient land to feed the world by the year 2050 using the current methods of livestock cultivation. See, Harry Aiking, “Future Protein Supply,” Trends in Food Science & Technology, March 2011, Vol. 22, Issues 2-3, Pages 112-120, the entire contents of which are hereby incorporated by reference in its entirety. This, coupled with the health concerns associated with meat consumption, has resulted in a shift to the consumption of plant-based food products. In fact, plant-based food products provide numerous benefits as compared to the animal-based food products they replace. For example, plant-based food products provide health benefits (e.g., less cholesterol or lower levels of saturated fats) and eliminate the negative aspects of animal husbandry, including the environmental impacts of such, the animal confinement, the disruption of maternal-offspring interactions, and the slaughter of animals for their meat.

Cell-based meat (CBM) production is a promising technology that could generate meat without the need for animal agriculture. The generation of tissue requires a three-dimensional (3D) scaffold to provide support to the cells and mimic the extracellular matrix (ECM). Numerous groups have developed methods to manufacture these scaffolds that support the production of environmentally friendly cultivated products, such as cell-cultured meats. Since algae is nutrient-rich and relatively easy and inexpensive to grow, some groups have used algae-based scaffolds to grow food products.

Since mycelium contains chitin, which can be made to mimic some of the polysaccharides found in the natural ECM, and some fungi have a meaty taste and texture, other groups have used fungal mycelium as a scaffolding substrate.

However, these plant and fungi-based scaffolds are just the beginning. What is needed is an enhanced method and system in the field of animal-free scaffolding that helps cell-cultured meat producers to cut costs and reduce their environmental impact. More specifically, improved methods are needed for creating edible scaffolding using unique and sustainable materials.

### Examples of Related Art are Described Below

KR102151203B1 describes a support for cell cultures, which has a hydrogel structure composed of cellulose and alginates extracted through the decellularization of seaweed. This application uses an ozone step in the decellularization process that is problematic. Ozone is very reactive and corrosive, thus requiring corrosion-resistant material, such as stainless steel. Ozone is extremely irritating and possibly toxic, so off-gases from the contactor must be destroyed to prevent worker exposure. This adds great cost and time to the process employed.

WO2017160862A1 describes decellularized plant tissues and the use of these plant tissues as scaffolds. Particularly, decellularized plant tissues are functionalized to allow for human cell adhesion, thereby allowing for their use as scaffolds for human cells. These scaffolds can then be used in a number of applications/markets, including as research tools for tissue engineering, regenerative medicine, and basic cellular biology.

CN111227199A describes a preparation method of ready-to-eat Durvillaea antarctica.

CN103637858A describes a manufacturing method of a three-dimensional porous scaffold in bioengineering, where a fiber structure of the scaffold includes gelatin, seaweed gel and collagen.

One group describes three animal-free scaffolds: decellularized plant tissue, chitin/chitosan and recombinant collagen scaffolds. Decellularized plant tissue provides a wide array of structures with varying biochemical, topographical and mechanical properties; chitin/chitosan-based scaffolds have shown synergistic bactericidal effects and improved cell-matrix interaction; and lastly, recombinant collagen has the potential to closely resemble native tissue, as opposed to the other two. These benefits, alongside potential scalability and tunability, open the door to applications beyond the biomedical realm, such as innovations in cellular agriculture and future food technologies. See, Santiago Campuzano, et al., “Scaffolds for 3D Cell Culture and Cellular Agriculture Applications Derived From Non-Animal Sources,” Front. Sustain. Food Syst. Mini Review, May 2019, Volume 3, Article 38, Pages 1-9, the entire contents of which is hereby incorporated by reference in its entirety.

Others have addressed different types of additives and their impact on various functional properties of seaweed based composites, their methods of incorporation, and applications with special emphasis on food and pharmaceutical usage. See, H. P. S. Abdul Khalil, et al., “Seaweed Based Sustainable Films and Composites for Food and Pharmaceutical Applications: A Review,” Renewable and Sustainable Energy Reviews, September 2017, Volume 77, Pages 353-362, the entire contents of which is hereby incorporated by reference in its entirety. Another group provides a review that summarizes and broadly classifies all of the major sustainable natural carbohydrate bio-macromolecular manifestations in nature—from botanical (cellulose, starch, and pectin), seaweed (alginate, carrageenan, and agar), microbial (bacterial cellulose, dextran, and pullulan), and animal (hyaluronan, heparin, chitin, and chitosan) sources—that have been contrived into electrospun fibers. See, K. P. Akshay Kumar, et al., “Electrospun Fibers Based on Botanical, Seaweed, Microbial, and Animal Sourced Biomacromolecules and their Multidimensional Applications,” International Journal of Biological Macromolecules, February 2021, Volume 171, Pages 130-149, the entire contents of which is hereby incorporated by reference in its entirety.

Others have developed an environmentally conscious, lean, structured meat product using decellularized plant leaf scaffold technology. See, Fatin Alkhaledi, et al., “Leef Jerky™: A Sustainable Meat Product for a Better Future,” The Worcester Polytechnic Institute, 2019, the entire contents of which is hereby incorporated by reference in its entirety. Another group has developed natural scaffolds in the form of decellularized amenity grass, which retains its natural striated topography and supports the attachment, proliferation, alignment and differentiation of murine C2C12 myoblasts, without the need for additional functionalization. See, Scott J. Allan, et al., “Decellularized Grass as a Sustainable Scaffold for Skeletal Muscle Tissue Engineering,” J. Biomed. Mater Res. A., 2021, 109(12), Pages 2471-2482, the entire contents of which is hereby incorporated by reference in its entirety.

Another group describes the utilization of green seaweed and its derived ulvan polysaccharides for the preparation of numerous chemicals (e.g., solvents, fuel, and gas) and also value-added biomaterials with various morphologies (e.g., gels, fibers, films, scaffolds, nanomaterials, and composites). See, D. Shanthana Lakshmi, et al., “A Short Review on the Valorization of Green Seaweeds and Ulvan: Feedstock for Chemicals and Biomaterials,” Biomolecules, 2020, 10(7), Page 991, the entire contents of which is hereby incorporated by reference in its entirety. One group describes the use of decellularization—recellularization approach to fabricate seaweed cellulose-based scaffolds for in vitro mammalian cell growth. See, Nurit Bar-Shai, et al., “Seaweed Cellulose Scaffolds Derived from Green Macroalgae for Tissue Engineering,” Scientific Reports, 11, Article Number 11843, the entire contents of which is hereby incorporated by reference in its entirety. Other groups describe decellularization of spinach leaves to produce an edible scaffold that has a vascular network that could potentially maintain the viability of primary bovine satellite cells as they develop into meat. See, Jordan D. Jones, et al., “Decellularized Spinach: An Edible Scaffold for Laboratory-Grown Meat,” Food Bioscience, June 2021, Volume 41, Article Number 100986, the entire contents of which is hereby incorporated by reference in its entirety.

Various similar systems exist in the art. However, their means of operation are substantially different from the present disclosure, as the other inventions fail to solve all the problems taught by the present disclosure.

### SUMMARY OF THE INVENTION

The present invention and its embodiments relate to methods for creating edible scaffolding using algae. More specifically, the present invention and its embodiments relate to a protocol for the sterilization and decellularization of Durvillaea antarctica, Chilean seaweed, or cochayuyo to prepare the cochayuyo to be suitable for the culture of cells.

An embodiment of the present invention describes a method to decellularize an algae to form a scaffold for cell seeding. Decellurization is the chemical, physical or enzymatic process of treating cells to remove components from the cell that will elicit an immune response. Accordingly, decellurization affords the cells the ability to be used in ECMs (extracellular matrices) without having to be concerned about eliciting an immune response. The instant method includes numerous process steps, such as: washing an algae with distilled water. In an embodiment, the algae is a species of brown algae and in a variation, is Durvillaea antarctica, Chilean seaweed, or cochayuyo. Next, the method includes chopping and grinding the algae and transferring a selected sample of the algae to a tube, adding the distilled water, and storing the selected sample of the algae at a first temperature (e.g., about 4° C.) for a first time period (e.g., overnight or 8-18 hours). Next, the method includes extracting the distilled water and adding a first solution (e.g., an SDS solution) to the tube at a second temperature (e.g., room temperature) for a second time period (e.g., five days).

Then, the method includes centrifuging the sample for a third time period (e.g., about five minutes), removing a supernatant, and washing with the distilled water. Next, the method includes adding a second solution (e.g., a CaCl₂ solution) to the tube and incubating the sample at a third temperature (e.g., about 4° C.) for a fourth time period (e.g., about twenty-four hours). Next, the method includes centrifuging the sample for a fifth time period (e.g., about 5 minutes) and removing the supernatant. Then, the method includes adding the distilled water to the tube and storing the sample at a fourth temperature (e.g., about 4° C.) for a sixth time period (e.g., overnight). Next, the method includes centrifuging the sample for a seventh time period (e.g., about five minutes), removing the supernatant, and storing the sample in a third solution (e.g., a PBS solution) at a fifth temperature until use (e.g., about −20° C.).

After freezing, the method further comprises thawing the sample in a thermal bath at a sixth temperature of about 40° C. for an eighth time period (e.g., in a range between about 5 minutes to about 10 minutes). Next, the method further includes centrifuging the sample for a ninth time period (e.g., about five minutes) and extracting the supernatant. Then, the method includes: drying the sample in an oven at a seventh temperature (e.g., about 60° C.) and for a tenth time period (e.g., about two hours). The method then includes: sterilizing the sample by autoclaving and transferring the sample, in a laminar flow hood, to tubes, adding a gelatin, and storing the sample at an eighth temperature (e.g., room temperature) for an eleventh time period (e.g., about twenty-four hours). Next, the method further includes: centrifuging the sample for a twelfth time period (e.g., about five minutes) and removing the supernatant in the laminar flow hood such that the scaffold is ready for cell seeding. The scaffold described herein is composed of sterile decellularized cochayuyo rehydrated with adhesion/carrier proteins (e.g., 1% w/v gelatin, 1% w/v albumin, and/or other molecules). Moreover, the scaffold is edible. The method may optionally further include assaying the scaffold and comparing the scaffold to collagen microcarriers in terms of cell growth.

### BRIEF DESCRIPTION OF THE SEVERAL VIEWS OF THE DRAWINGS

FIG. 1 depicts a block diagram of a method for creating an edible scaffold using Durvillaea antarctica, Chilean seaweed, or cochayuyo, in accordance with embodiments of the present invention.

FIG. 2 depicts a block diagram of a method associated with use of the product of FIG. 1 after freezing, in accordance with embodiments of the present invention.

FIG. 3 shows Staining of Durvillaea antarctica during scaffold manufacture. a) Crushed cochayuyo. b) Cochayuyo after 5 days of decellularization with SDS. c) Cochayuyo after neutralization process.

FIG. 4 shows DNA concentration in the SDS supernatant during the decellularization period of Durvillaea antarctica.

FIG. 5 shows Concentration of genetic material in Durvillaea antarctica before and after the complete process

FIG. 6A and FIG. 6D show cellularized cochayuyo (untreated)×10. FIG. 6B and FIG. 6E show 5 days of decellularization with SDS×10. FIG. 6C and FIG. 6F show after neutralization process×10. The top row shows brown algal structure, and the bottom row shows decellularization level of Durvillaea antarctica (compression with coverslip). Cells are enclosed in a red circle, while hollows are shown in yellow circles.

FIG. 7A shows Cellularized cochayuyo (without treatment). FIG. 7B and FIG. 7C show Cochayuyo after decellularization process with SDS. All show SEM images (FEI Quanta 250) of the structure of dried Durvillaea antarctica.

FIG. 8 shows Plate culture of C2C12. a) 2 hours of ×10 culture. b) 26 hours of ×10 culture. c) 100 hours of culture×10 (augmentation)

FIG. 9A shows Plate culture of C2C12 with Growth curve. FIG. 9B shows consumption and generation of metabolites.

FIG. 10A shows specific rates of glucose consumption and FIG. 10B shows specific rate of lactate generation culture of C2C12 in plates.

FIG. 11 shows cellular density of C2C12 on plates at different times of culture.

FIG. 12 shows C2C12 culture on Durvillaea antarctica scaffolds on plates. a) 2 hours of ×10 culture. b) 73 hours of ×10 culture. c) 122 hours of ×10 culture. d) 168 hours of ×10 culture. ×10 culture.

FIG. 13A shows C2C12 culture on Durvillaea antarctica scaffolds on plates with the growth curve and FIG. 13B shows Consumption and generation of metabolites.

FIG. 14A shows specific rates of metabolite generation or consumption, C2C12 culture on Durvillaea antarctica scaffolds on plates of Glucose consumption and FIG. 14B shows lactate generation.

FIG. 15 shows Cell density of C2C12 culture on Durvillaea antarctica scaffolds on a plate.

FIG. 16A shows Comparison of the maximum biomass concentration achieved in plate and scaffold cultures of Durvillaea antarctica Cell concentration p<0.05 and FIG. 16B shows Cell density p<0.05.

FIG. 17A shows C2C12 culture in microcarriers showing a growth curve and FIG. 17B shows consumption and generation of metabolites.

FIG. 18A shows Specific rates of metabolite generation or consumption, C2C12 culture in microcarriers of glucose consumption and FIG. 18B shows lactate generation.

FIG. 19 shows Cell density of C2C12 culture in microcarriers.

FIG. 20 shows Culture of C2C12 on Durvillaea antarctica scaffolds in suspension. a) 16 hours of ×10 culture. b) 70 hours of ×10 culture. c) 116 hours of ×10 culture. d) 164 hours of ×10 culture.

FIG. 21A shows C2C12 culture on Durvillaea antarctica scaffolds in suspension of the growth curve and FIG. 21B shows consumption and generation of metabolites.

FIG. 22A shows Specific rates of metabolite generation or consumption, C2C12 culture on suspended Durvillaea antarctica scaffolds in suspension showing glucose consumption and FIG. 22B shows lactate generation.

FIG. 23 shows Cell density of C2C12 culture on Durvillaea antarctica scaffolds in suspension.

FIG. 24A shows comparison of maximum biomass concentration achieved in microcarrier and scaffold cultures of Durvillaea antarctica in suspension for a cell concentration, t-student P<0.05 and FIG. 24B shows cell density t-student P>0.05.

FIG. 25 shows DNA contained in the SDS supernatant during the decellularization period of Ulva.

FIG. 26 shows DNA Concentration in Ulva rigida before and after the complete process. *P<0.05

FIG. 27 shows Photographs of ulva at three different stages of decellularization. a) Cellularized ulva, staining with crystal violet ×10. b) Ulva after 3 days of decellularization with SDS 1%, staining with crystal violet ×10. c) Ulva after neutralization, staining with crystal violet ×10.

FIG. 28 shows C2C12 culture on Ulva rigida scaffolds in suspension. a) 7 hours of culture ×10. b) 71 hours of culture ×10. c) 120 hours of culture ×10.

FIG. 29A shows Culture of C2C12 on Ulva rigida scaffolds in suspension of a growth curve and FIG. 29B shows consumption and generation of metabolites.

FIG. 30A shows specific metabolite generation or consumption rates, C2C12 culture on Ulva rigida scaffolds in suspension of glucose consumption and FIG. 30B shows lactate generation.

FIG. 31 shows Cell density of C2C12 culture on Ulva rigida scaffolds in suspension.

FIG. 32A shows comparison of maximum biomass concentration achieved in cultures on Ulva rigida scaffolds and on Durvillaea antarctica scaffolds in suspension cell concentration P<0.05 and FIG. 32B shows maximum cell density.

FIG. 33A shows comparison of maximum biomass concentration achieved in cultures on Ulva rigida scaffolds, on Durvillaea antarctica scaffolds and microcarriers in suspension for cell concentration and FIG. 33B shows cell density.

### DETAILED DESCRIPTION

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

In native tissues, the ECM provides structural support for cell growth and stimulates tissue regeneration. More specifically, the ECM is an intricate network composed of an array of multidomain macromolecules organized in a cell/tissue-specific manner. Components of the ECM link together to form a structurally stable composite, contributing to the mechanical properties of tissues. See, Beatrice Yue, “Biology of the Extracellular Matrix: An Overview,” J. Glaucoma, 2014, Pages S20-S23, the entire contents of which is hereby incorporated by reference in its entirety. Researchers are currently investigating unique methods to create microenvironments that mimic the biochemical and physiological structures of natural environments within the human body. Numerous fabrication strategies have been investigated for this purpose. As an illustrative example, cellulose-based matrices have been used to facilitate mammalian cell culture in vitro and in vivo. See, R. J. Hickey, et al., “Cellulose Biomaterials for Tissue Engineering,” Front. Bioeng. Biotechnol., 2019, the entire contents of which is hereby incorporated by reference in its entirety.

Cellulose is the most abundant polymer in nature and is a key structural element in the cell wall of plants. Specifically, cellulose is a polysaccharide consisting of a linear chain of several hundred to many thousands of β linked D-glucose units. With lignin and hemicellulose, cellulose supports plant's vertical growth. See, D. Klemm, et al., “Cellulose: Fascinating Biopolymer and Sustainable Raw Material,” Angew. Chemie Int. Ed., 2005, Vol. 44, Pages 3358-93; and L. J. Gibson, “The Hierarchical Structure and Mechanics of Plant Materials,” J. R. Soc. Interface, 2012, Vol. 9, Pages 2749-66, the entire contents of which are hereby incorporated by reference in their entireties. Due to the unique biophysical and biomechanical properties of cellulose, it may be used as a construct to guide the restructuring of cells and newly formed tissue for various applications. See, R. J. Hickey, et al., “Cellulose Biomaterials for Tissue Engineering,” Front. Bioeng. Biotechnol.,” 2019, Vol. 7, doi: 10.3389/fbioe.2019.00045, the entire contents of which is hereby incorporated by reference in its entirety.

Cellulose sources in tissue engineering include natural polymers derived from plants and synthetically-modified polymers. See, D. J. Modulevsky, et al., “Apple Derived Cellulose Scaffolds for 3D Mammalian Cell Culture,” PLoS ONE, 2014, Vol. 9, 97835; D. J. Modulevsky, et al., “Biocompatibility of Subcutaneously Implanted Plant-Derived Cellulose Biomaterials,” PLoS ONE, 2016, Vol. 11, e0157894; L. Fu, et al., “Present Status and Applications of Bacterial Cellulose-Based Materials for Skin Tissue Repair,” Carbohydr. Polym., 2013, Vol. 92, Pages 1432-42; and M. N. Nosar, et al., “Characterization of Wet-Electrospun Cellulose Acetate Based 3-Dimensional Scaffolds for Skin Tissue Engineering Applications: Influence of Cellulose Acetate Concentration,” Cellulose, 2016, Vol. 23, Pages 3239-48, the entire contents of which are hereby incorporated by reference in their entireties.

Seaweed, or macroalgae, refers to species of macroscopic, multicellular, marine algae. This term includes some types of Rhodophyta (red), Phaeophyta (brown) and Chlorophyta (green) macroalgae. Macroalgae has unique properties, including a high degree of cellulose crystallinity. See, D. Klemm, et al., “Cellulose: Fascinating Biopolymer and Sustainable Raw Material,” Angew. Chemie Int. Ed., 2005, Vol. 44, Pages 3358-93; and A. Mihranyan, “Cellulose from Cladophorales Green Algae: from Environmental Problem to High-Tech Composite Materials,” Polym. Polym. Compos., 2011, Vol. 119, Pages 2449-60, the entire contents of which are hereby incorporated by reference in their entirety. Specifically, green macroalgae's matrix consist of a highly robust skeleton structure that can be utilized for cell growth. See, M. Lahaye, et al., “Structure and Functional Properties of Ulvan, a Polysaccharide from Green Seaweeds,” Biomacromolecule, 2007, Vol. 8, Pages 1765-74, the entire contents of which are hereby incorporated by reference in their entirety.

Moreover, macroalgae have high growth rates and are abundant, making its mass production affordable. See, A. Chemodanov, et al., “Design of Marine Macroalgae Photobioreactor Integrated into Building to Support Seagriculture for Biorefinery and Bioeconomy,” Bioresour. Technol., 2017, Vol. 241, Pages 1084-93, the entire contents of which is hereby incorporated by reference in its entirety. Additionally, macroalgae has the added benefits of not competing with the food supply, land for agriculture and forestry, or the freshwater supply. See, F. Fernand, et al., “Offshore Macroalgae Biomass for Bioenergy Production: Environmental Aspects, Technological Achievements and Challenges,” Renew. Sustain. Energy Rev., 2017, Vol. 75, Pages 35-45, the entire contents of which is hereby incorporated by reference in its entirety. In fact, green macroalgae derived sulfated polysaccharides (SPs) have been proposed for tissue engineering purposes. See, P. Sudha, et al., “Ulvan in Tissue Engineering,” in Encyclopedia of Marine Biotechnology (ed. Kim, S.) 1335-1350 (Wiley, 2020), the entire contents of which is hereby incorporated by reference in its entirety. For at least the aforementioned reasons, cellulose-based ECM and macroalgae-based cellulose are becoming increasingly popular.

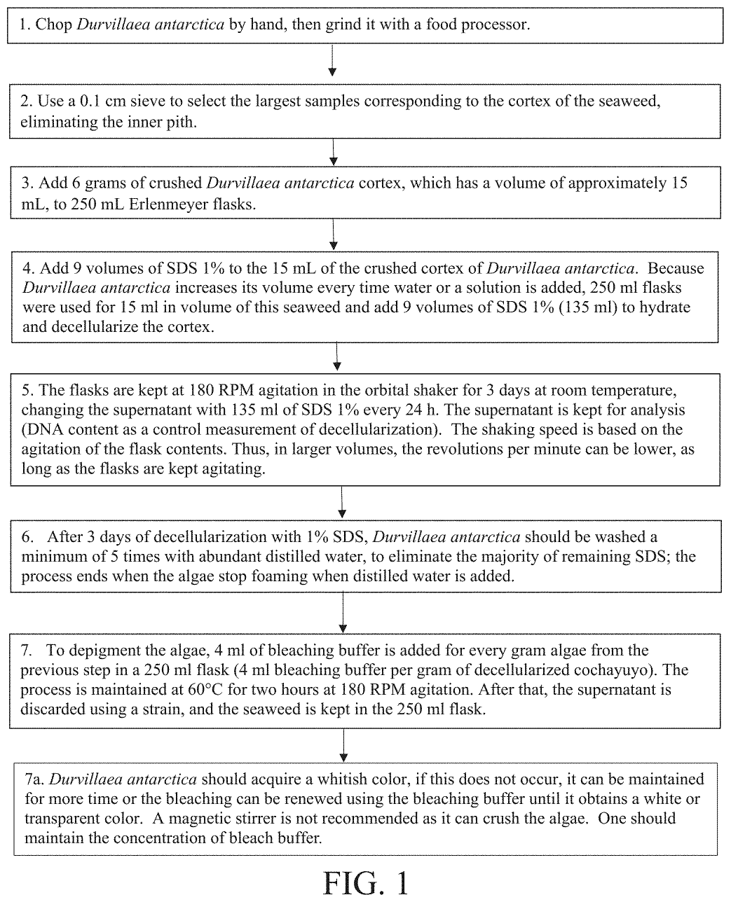

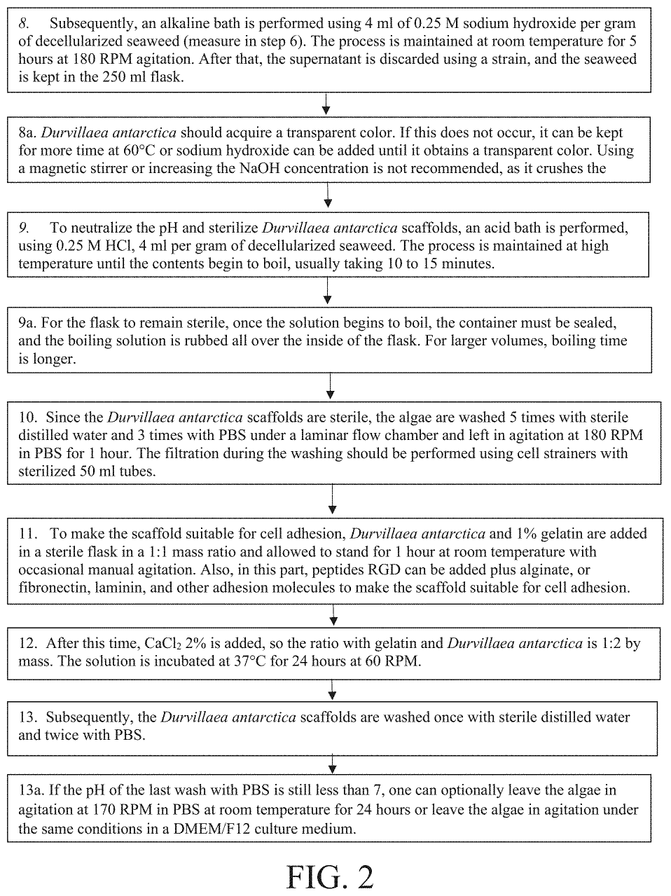

The present invention, as described herein, relates to the use of seaweed and methodology associated with scaffold generation. A method for creating an edible scaffold using an algae (e.g., Durvillaea antarctica, Chilean seaweed, or cochayuyo) is described and depicted in FIG. 1 and FIG. 2 herein. More specifically, the method of FIG. 1 and FIG. 2 is a protocol for the sterilization and decellularization of cochayuyo to prepare the seaweed to be suitable for the culture of cells. Durvillaea antarctica is a species of brown algae in the family Durvillaeaceae. D. antarctica is large, robust, and is found off the coasts of Chile and southern New Zealand. In fact, D. antarctica is an edible algae safe for consumption and in Chile, D. antarctica is eaten as a salad or stew post-hydration since this seaweed is sundried and sold as a dry food after its harvest. Moreover, D. antarctica has gained popularity, since it is inexpensive and is sustainable, unlike other plant-based scaffoldings. Moreover, D. antarctica can be easily cultivated and sterilized. After scaffolding, the species retains the insoluble fiber and some properties, making it a superfood. The final composition after decellularization is: total fibers 10%, proteins 0.2% and humidity is 90%.

The method of FIG. 1 begins at a process step 1 includes chopping the piece of cochayuyo by hand and then grinding the piece of cochayuyo (e.g., in a food processor or a similar device). A process step 2 follows the process step 1 and includes using a 1-millimeter sieve to select the largest samples of the cochayuyo. A process step 3 includes transferring the selected samples to a flask (e.g., a 250 mL Erlenmeyer flasks):

A process step 4 follows the process step 3 and includes adding a first solution (e.g., adding 9 volumes of 1% w/v SDS solution) for a first time period (e.g., about three to five days) and at a first temperature (e.g., room temperature). The solution may be refreshed every twenty-four hours. It should be appreciated that the SDS solution is sodium dodecyl sulfate in distilled, deionized water.

A process step 5 follows the process step 4 and includes keeping the flask in agitation (e.g. 180 rpm) and also includes centrifuging the sample at about 1500 rpm for a second time period (e.g., about 5 minutes). Next, the process step includes removing the supernatant. A process step 6 follows the process step 5 and includes washing with distilled water five times and checking for foam reduction.

A process step 7 follows the process step 6 and includes adding a second solution (e.g., 4 ml bleaching buffer (sodium acetate, acetic acid, and sodium chlorite solution) per gram of decellularized cochayuyo) and incubating the sample for a third time period (e.g., about two hours) and at a second temperature (e.g., about 60° C.) in agitation (e.g. 180 rpm). A process step 7 includes bleaching of the sample and ends the method of FIG. 1.

FIG. 2 depicts numerous process steps associated with use of the sample product of FIG. 1 after bleaching. A process step 8 follows the process step 7 and includes adding a third solution: alkaline bath (e.g. 4 ml of 0.25 M sodium hydroxide per gram of decellularized seaweed) and incubating the sample for a fourth time period (e.g., about two hours) and at a second temperature (e.g., about 60° C.) in agitation (e.g. 180 rpm).

Then, a process step 9 follows the process step 8 and includes adding a fourth solution: an acid bath to neutralize the pH and sterilize the sample (e.g. 4 ml of 0.25 M hydrochloric acid per gram of decellularized seaweed) and incubating the sample for a fifth time period (e.g., 10 to 15 minutes) and at a fourth temperature (e.g., boiling temperature). The supernatant is then removed and process step 9 includes adding the distilled water to the tube and storing the sample at a fifth temperature (e.g. 4° C.) until use.

Subsequently in a laminar flow hood, a process step 10 follows the process step 9 and includes thawing the sample in sterilized water at a sixth temperature (e.g. room temperature) for a seventh time period (e.g., 5 to 10 minutes) for five times. The process step 10 also includes adding a fifth solution (e.g. PBS) and incubating the sample for a seventh temperature (e.g., room temperature) and a ninth time period (e.g. 1 hour).

Then, in a laminar flow hood, a process step 11 follows the process step 10 and includes transferring the sterile cochayuyo to flasks (e.g., 50 mL tubes), adding about 1 volume of sterile gelatin or albumin, fibronectin or peptides RGD (e.g. 0.1 to 5%) allowing it to stand for a tenth time period (e.g. 1 hour) at room temperature, preferentially.

A process step 12 follows the process step 11 and includes adding a sixth solution (e.g. a 1:2 ratio of gelatin relative to a sterilized CaCl₂) 2% solution), and then storing the sample at an eighth temperature (e.g., at about 37° C.) for an eleventh time period (e.g. 24 hours). Then, the supernatant is removed in the laminar flow hood. Now, the scaffolding is ready for cell seeding. A process step 13 follows the process step 12 to conclude the method of FIG. 2.

The final product of the method of FIG. 2 is a scaffold for cell growth composed of sterile decellularized cochayuyo rehydrated with adhesion/carrier proteins, such as 1% w/v gelatin, 1% w/v albumin, RGD peptides or other molecules. The decellularized algae supports cell growth, differentiation, and nutrient diffusion due to its porosity. The product is completely edible because none of the methodologies of decellularization described herein involve the use of toxic compounds. See, J. Viitala, et al., “Sodium Dodecyl Sulfate (SDS) Residue Analysis of Foam Formed Cellulose-Based Products,” Nordic Pulp & Paper Research Journal, 2020, Vol. 35, Issue 2, Pages 261-271, the entire contents of which is hereby incorporated by reference in its entirety.

The methods of the present invention differ from other edible scaffolding methods or techniques known in the art field due to the scalability, cost, and the ways in which the seaweed material can be dried and rehydrated without problems before and/or after decellularization. Also, the sample described herein can be easily sterilized without losing its form and can be hydrated with proteins. Furthermore, the scaffolding described herein can be used as a microcarrier because it can float inside the cell media, and one does not need to change the cells for differentiation.

### Examples

Procedure for Decellularization of Durvillaea antarctica (Cochayuyo)

This protocol was also used in the decellularization of Pelillo (gracilaria chilensis), Luche (Pyropia columbina), and Ulva (Ulva rigida), which are several Chilean algae, without modification, and the C2C12 cells growth were tested using Ulva scaffolding.

The following procedure refers to cochayuyo only, but it should be understood that it will work with the other seaweeds enumerated herein.

The following materials were procured:
Sodium dodecyl sulfate (SDS) (Merck, 428015)
Sodium chlorite (NaClO2) (Sigma, 244155)
Sodium hydroxide (NaOH) (Panreac, 131687)
Hydrochloric acid (HCl 37%) (Panreac, 131020)
Citric acid (1-Hydrate) (Winkler, AC-0060)
Triton X-100 (Sigma, X100)
Sodium acetate (Winkler, SO-1395)
Calcium dichloride (CaCl2) (Winkler, CA-0520)
Acetic acid (90%) (Winkler, 100180)
Gelatin food grade (bought at the supermarket)
Crystal violet (Merck, 101408)
Phosphate buffered saline (PBS) (Gibco, 21600-010)
Durvillaea antarctica (bought at the supermarket)
Liquid nitrogen
Hexadecyltrimethylammonium bromide (CTAB) (MP biomedical s, 208232)
Polyvinylpyrrolidone (PVP) (Sigma, PVP-40)
Titriplex (EDTA) (Merck, 108418)
Tris (Winkler, BM-2000)
Sodium chloride (NaCl) (Panreac, 131659)
Sterile distilled water
PCR-grade water (Sigma, 3315959001)
   The following equipment was procured:
Orbital shaker (Thermo Scientific, SHKE2000002)
MaestroNano Micro-Volume Spectrophotometer (MaestroGen, MN-913)
Microscope (Leica, DM500)
Incubator (Shel Lab, TC2323)
Food processor (Thomas, TH-9005V)
Laminar flow chamber (Nuare, Nu201330E)
pH meter (HANNA)
Eppendorf centrifuge (Dlab, D3024)
   The following solutions were prepared:
   500 mL of 10% w/v SDS using 50 g SDS and 450 mL distilled water
   500 mL of SDS 1% w/v was made using 50 mL SDS 10% w/v and 450 mL distilled water
   1 L PBS was made using one sachet of PBS was added to 1 L of distilled water at pH 7.30 and was autoclaved.
   100 mL crystal violet was prepared using 0.10 g crystal violet, 2.10 g citric acid and 1 mL Triton X-100
   100 mL gelatin 1% w/v was prepared using 1 g gelatin, 99 mL distilled water at pH 7.30 and was autoclave
   100 mL bleaching buffer was made using 812 mg sodium acetate, 625 μL acetic acid, 234 mg sodium chlorite, and distilled water was added to make 100 mL of solution at pH 4.60.
   100 mL sodium hydroxide 0.25 M was made using 1 g sodium hydroxide and distilled water was added to make a 100 mL solution.
   100 mL of a 0.25 M hydrochloric acid was made using 205 mL hydrochloric acid and distilled water was added to make 100 mL of solution.
   50 mL CaCl2 2% w/v was made using 1 g CaCl₂), and distilled water was added to 50 mL. The solution was filtered with 0.22 μm pyrinol filters.
   50 mL DNA extraction buffer was made using 1 g CTAB,1 g PVP, 4.10 g NaCl, 2 mL EDTA 05 M pH 8.00, 10 mL 0.05 M Tris pH 9 Make up to 100 mL with PCR-grade water, and the solution was filtered with 0.22 μm filters
   Procedure for the Generation of Durvillaea antarctica (Cochayuyo) Scaffolds/Microcarriers

The Cochayuyo's scaffoldings are superior because they function better as microcarriers in suspension cultures relative to other scaffolds.
1. Chop Durvillaea antarctica by hand, then grind it with a food processor.
2. Use a 0.1 cm sieve to select the largest samples corresponding to the cortex of the seaweed, eliminating the inner pith.
3. Add 6 grams of crushed Durvillaea antarctica cortex, which has a volume of approximately 15 mL, to 250 mL Erlenmeyer flasks.
4. Add 9 volumes of SDS 1% to the 15 mL of the crushed cortex of Durvillaea antarctica.
   Because Durvillaea antarctica increases its volume every time water or a solution is added, 250 ml flasks were used for 15 ml in volume of this seaweed and add 9 volumes of SDS 1% (135 ml) to hydrate and decellularize the cortex.
5. The flasks are kept at 180 RPM agitation in the orbital shaker for 3 days at room temperature, changing the supernatant with 135 ml of SDS 1% every 24 h. The supernatant is kept for analysis (DNA content as a control measurement of decellularization).
   The shaking speed is based on the agitation of the flask contents. Thus, in larger volumes, the revolutions per minute can be lower, as long as the flasks are kept agitating.
6. After 3 days of decellularization with 1% SDS, Durvillaea antarctica should be washed a minimum of 5 times with abundant distilled water, to eliminate the majority of remaining SDS; the process ends when the algae stop foaming when distilled water is added.
7. To depigment the algae, 4 ml of bleaching buffer is added for every gram algae from the previous step in a 250 ml flask (4 ml bleaching buffer per gram of decellularized cochayuyo). The process is maintained at 60° C. for two hours at 180 RPM agitation. After that, the supernatant is discarded using a strain, and the seaweed is kept in the 250 ml flask.
   Durvillaea antarctica should acquire a whitish color, if this does not occur, it can be maintained for more time or the bleaching can be renewed using the bleaching buffer until it obtains a white or transparent color. A magnetic stirrer is not recommended as it can crush the algae. One should maintain the concentration of bleach buffer.
8. Subsequently, an alkaline bath is performed using 4 ml of 0.25 M sodium hydroxide per gram of decellularized seaweed (measure in step 6). The process is maintained at room temperature for 5 hours at 180 RPM agitation. After that, the supernatant is discarded using a strain, and the seaweed is kept in the 250 ml flask.
   Durvillaea antarctica should acquire a transparent color. If this does not occur, it can be kept for more time at 60° C. or sodium hydroxide can be added until it obtains a transparent color. Using a magnetic stirrer or increasing the NaOH concentration is not recommended, as it crushes the algae.
9. To neutralize the pH and sterilize Durvillaea antarctica scaffolds, an acid bath is performed, using 0.25 M HCl, 4 ml per gram of decellularized seaweed. The process is maintained at high temperature until the contents begin to boil, usually taking 10 to 15 minutes.
   For the flask to remain sterile, once the solution begins to boil, the container must be sealed, and the boiling solution is rubbed all over the inside of the flask. For larger volumes, the boiling time is longer.
10. Since the Durvillaea antarctica scaffolds are sterile, the algae are washed 5 times with sterile distilled water and 3 times with PBS under a laminar flow chamber and left in agitation at 180 RPM in PBS for 1 hour. The filtration during the washing should be performed using cell strainers with sterilized 50 ml tubes.
11. To make the scaffold suitable for cell adhesion, Durvillaea antarctica and 1% gelatin are added in a sterile flask in a 1:1 mass ratio and allowed to stand for 1 hour at room temperature with occasional manual agitation. Also, in this part, peptides RGD can be added plus alginate, or fibronectin, laminin, and other adhesion molecules to make the scaffold suitable for cell adhesion.
12. After this time, CaCl₂) 2% is added, so that the ratio with gelatin and Durvillaea antarctica is 1:2 by mass. The solution is left incubating at 37° C. for 24 hours at 60 RPM.
13. Subsequently, the Durvillaea antarctica scaffolds are washed once with sterile distilled water and twice with PBS.
   If the pH of the last wash with PBS is still less than 7, one can optionally leave the algae in agitation at 170 RPM in PBS at room temperature for 24 hours or leave the algae in agitation under the same conditions in a DMEM/F12 culture medium.
   Procedure for Decellularization Characterization of Durvillaea antarctica
1. From the decellularization of Durvillaea antarctica with 1% SDS, the supernatant was recovered daily, DNA content was quantified using in triplicate 1 μL of the supernatant, corresponding to the 3 days in the MaestroNano equipment, using the 1% SDS solution as a blank and an absorbance measurement at a wavelength of 260 nm. The results were recorded in a graph of DNA concentration vs. days of decellularization.
   To obtain more accurate measurements, it is recommended that the container that stores the supernatant be free of foam.
2. The degree of decellularization of cellularized cochayuyo samples is compared with Durvillaea antarctica specimens, from each stage of the decellularization and bleaching processes, using a Leica microscope. In both cases, the cells are stained with 20 μL of crystal violet, which are placed on a slide. First, the intact structure of the scaffold is photographed with the Leica microscope and then the degree of decellularization is captured photographically by compressing the algae with a coverslip. At the same time, each stage of the process is photographed, in a macro scale to record the bleaching of the algae.
   Photographs under the microscope should be obtained quickly, since the violet crystal dries, producing a dehydrated algae that has a changed structure.
3. To quantify the degree of decellularization, after neutralization with HCl, 0.5 grams of Durvillaea antarctica post-treatment and 0.5 grams of the cellularized algae are weighed in triplicate. The samples are placed in a mortar, and 15 mL of liquid nitrogen is added. The pieces are crushed in a mortar and pestle. Then, 1 mL of direct DNA extraction buffer is added to the crushed samples, and as much of the crushed sample as possible is recovered in 1.5 mL Eppendorf tubes. The tubes are centrifuged for 5 min at 13000 RPM (16089×g) and the DNA concentration is measured using 1 μL of each tube in the MaestroNano instrument at 260 nm using the DNA extraction buffer as a blank. The results are recorded in a graph of DNA concentration vs. treatment condition of Durvillaea antarctica.

The following protocols lay out how to perform a cell culture on Durvillaea antarctica scaffolds.

C2C12 cell culture on Durvillaea antarctica scaffolds.

The below overall protocol can also be used for primary cultures of myoblasts or every type of mammalian cell, including stem cells and cells suitable for cultivated meat.

The following materials were used:
Cell viability kit with MTT (ThermoFisher, V13154).
Hydrochloric acid (HCl 37%) (Panreac, 131020).
Crystal violet (Merck, 101408).
Citric acid (1-Hydrate) (Winkler, AC-0060).
Triton X-100 (Sigma, X100).
Phosphate-buffered saline (PBS) (Gibco, 21600-010).
High-glucose DMEM medium (Gibco, 12100-046).
Ham's F12 nutrient mixture (Gibco, 21700-075).
Fetal bovine serum (FBS) (Biosera, FB-1001).
Horse serum (HS) (Gibco, 26050088).
Sigmacote (Sigma, SL2).
Trypan blue (Sigma, T0776).
Trypsin (10×) (Gibco, 15400-054).
Durvillaea antarctica scaffolds.
C2C12 cell line passage 13.
Sodium bicarbonate (Sigma, S4019).
CO2 cylinder (Linde).
Glucose measurement kit (Biosystems, 12800).
Lactate measurement kit (Biosystems, 12802).

The following equipment was used:
SPECTROstar Omega plate reader (BMG labtech).
Neubauer chamber.
Thermal bath (Labtech).
Microscope (Leica, DM500).
Incubator (Shel Lab, TC2323).

Magnetic stirrer with Cimarec Biosystem 40B controller (Thermo Scientific, 50087904).
Automatic analyzer in Random Access (Biosystem, Y15).
Laminar flow chamber (Nuare, Nu201330E).
Microscope (Olympus, CK2).
pH meter (HANNA).
50 mL tube centrifuge (Boeco, C-28).
Vortex (FinePCR, FineVortex).
Mini-centrifuge (Thermo Scientific, mySPIN 6).

The following solutions were prepared prior to initiating the procedure:

### PBS

1 L PBS using 1 sachet of PBS, and up to 1 L with distilled water was made at pH 7.30. Autoclave the PBS

### 100 mL Crystal Violet

100 mL crystal violet was made using 0.10 g crystal violet, 2.10 g citric acid, and 1 mL Triton X-100.

### Trypan Blue

100 mL trypan blue was made using 0.4 g trypan blue up to 100 mL using PBS

### Trypsin

10 mL trypsin (1×) was made using 1 mL trypsin (10×) and up to 10 mL PBS

2 L DMEM/F12 10% FBS culture medium was made using the following:

1 envelope DMEM high glucose medium

1 sachet Ham's F12 nutrient mix

4.01 g sodium bicarbonate

Make up to 2 L with water milli Q

pH 7.30

Filter through 0.22 μm membrane filter

200 mL FBS

10 mL 0.01 M HCl was made using the following:

8 μL HCl

Make up to 10 mL with distilled water.

1 mL 12 mM MTT

1 vial MTT from cell viability kit with MTT

1 mL PBS

Filtrate with 0.22 μm pyrindole filters.

10 mL MTT arrest solution was made using the following:

1 tube of SDS from the cell viability kit with MTT

10 mL 0.01 M HCl

Heat in thermal bath

2 L DMEM/F12 2% HS culture medium was made using the following:

1 sachet DMEM high glucose medium

1 sachet Ham's F12 nutrient mix

4.01 g sodium bicarbonate

Make up to 2 L with water milli Q

pH 7.30

Filter through 0.22 μm membrane filter

40 mL HS

### C2C12 Cell Thawing Procedure

To a T-flasks 175 culture plate, add 33 mL of DMEM/F12 10% FBS culture medium.

To a cryovial containing 1 mL frozen C2C12 cells, add 1 mL of DMEM/F12 10% FBS culture medium until the contents of the vial are melted.

This process must be done quickly, since the DMSO in which the cells are frozen is toxic to them. Add the thawed 2 mL contained in the cryovial to the T-flasks 175 culture plate. Leave the culture plate in the incubator at 37° C. and 5% CO2 until 70-80% confluence is reached.

### Procedure for Passage of C2C12 Cells

In a T-flask 175 culture plate, remove all the culture medium. Wash the plate 3 times with PBS. It is important to remove all the culture medium and all the PBS. For this, the plate can be left in a vertical position, so that all the liquids can be removed. Add 1 mL of 1× trypsin and incubate for 10 minutes at 37° C. Subsequently, stop the protease with 10 mL of fresh culture medium and extract a 200 μL aliquot in case a cell count is required. Place the cell solution in a 50 mL tube and centrifuged at 1000 RPM (160×g) for 5 minutes. Discard the culture medium by inversion. Dilute the pellet in 6 mL of DMEM/F12 10% FBS medium.

To 3 T-flasks 175 culture plates, add 33 mL of DMEM/F12 10% FBS medium. To each plate, add 2 mL of the pellet previously resuspended in culture medium. The culture plates are incubated at 37° C. and 5% CO2 until 70-80% confluence is reached.

Procedure for Making a Viability Calibration Curve with MTT

In a T-flasks 175 culture plate, remove all the culture medium. Wash the flasks 3 times with PBS. Add 1 mL of 1×trypsin and incubate 10 minutes at 37° C. Subsequently, stop the protease with 10 mL of fresh culture medium. Place the cell solution in a 50 mL tube and centrifuged at 1000 RPM (160×g) for 5 minutes. In parallel, count cells using a 200 μL aliquot Discard the culture medium by inversion. The pellet is diluted in FBS-free DMEM/F12 medium such that 3.00 ×10⁴ cells are obtained in 100 μL. Take 100 μL in duplicate and place in wells in a 96-well plate. It is important to resuspend the cells correctly so that the cell concentration is as accurate as possible. Subsequently, dilute the solution to obtain 2.50×10⁴ cells in 100 μL. Then, take 100 μL in duplicate and place in wells in a 96-well plate.

The process should be repeated until the following 9 points for the calibration curve are obtained in duplicate: 3,00×10⁴, 2,50 ×10⁴, 2,00 ×10⁴, 1,50 ×10⁴, 1,00 ×10⁴, 0,50 ×10⁴, 0,25 ×10⁴, 0,10 ×10⁴ and 0 cells.

Subsequently, 10 μL of 12 mM MTT is added to each well of the plate and left incubating at 37° C. for 4 hours. Once the time period has elapsed, 100 μL of the MTT stop solution is added to each well and the plate is incubated at 37° C. for 4 hours. Then, MTT stop solution is added to each well and incubated at 37° C. for 4 to 18 hours. Finally, the absorbance of each well is measured at a wavelength of 570 nm on the SPECTROstar Omega plate reader.

The results are recorded on a graph of absorbance vs. number of cells, to which a linear fit is made to obtain a lineal equation.

Procedure for Culture on Scaffolds of Durvillaea antarctica on a Plate

Remove all the media of a T-flasks 175 culture plate. Wash the plate 3 times with PBS. Add 1 mL of 1× trypsin and incubate 10 minutes at 37° C. Subsequently, stop the protease with 10 mL of fresh culture medium and take 200 μL aliquot for cell counting. Place the cell solution in a 50 mL tube and centrifuge at 1000 RPM (160×g) for 5 minutes. In parallel, count cells using the 200 μL aliquot. Discard the culture medium by inversion. The pellet is diluted in such a way that in 10 μL 5.00×10⁴ cells are obtained.

In parallel, 0.5 cm2 pieces of Durvillaea antarctica scaffolds are deposited in wells of a 24-well plate previously siliconized with sigmacote. Specifically, for each day of culture it is recommended to perform a septuplicate. So, each day, take samples from one well per day, cell media for analysis and a scaffolding for pictures. To siliconize with sigmacote, 100 μL of sigmacote is added to a well and the liquid is spread over the entire surface, then the 100 μL are transferred to another well to perform the same process. Once all the wells to be used have been siliconized, the plate should be left under UV light until all the remaining sigmacote evaporates.

To each piece of Durvillaea antarctica add 10 μL of the concentrated cell solution, leaving it to incubate for 40 minutes at 37° C. to promote cell adhesion to the scaffold. Once the 40 minutes have elapsed, the scaffold pieces are transferred to other 24-well plates, previously siliconized, and 1 mL of DMEM/F12 10% FBS culture medium is added. The plates are maintained at 37.0 and 5% CO2 during 7 days and each day one sample is taken for scaffolding pictures and glucose/lactate measurements.

Procedure to Characterize Scaffold Culture of Durvillaea antarctica in Plates.

To the culture made in plates using scaffoldings, do the following: Daily, take wells from the culture previously made. Keep the culture media. The scaffoldings will be helpful to make several analyses. Subsequently, wash the well with PBS twice

First, the morphology was evaluated using crystal violet and pictures taken with a microscope.

The following procedure was followed: Take the scaffolding with a tweezer and put it in a glass slide. Add 20 μl of crystal violet and soak the scaffolding with it and then remove the excess of crystal violet. Take pictures with microscope. The pictures should be taken quickly because the crystal violet dries, producing a dehydration of the algae that changes its structure.

Subsequently, the nuclei were counted using crystal violet: Three samples of Durvillaea antarctica scaffolds were introduced into 3 Eppendorf tubes, 40 μL of crystal violet is added and incubated at 37° C. for 1 hour. Subsequently, the scaffolds are vortexed at maximum power for 15 seconds, then spun in the minicentrifuge, 10 μL are extracted and deposited in a Neubauer chamber and cell counting is performed. In addition, the pieces of cochayuyo were measured with a ruler. If the cells do not detach from the scaffolds after 15 seconds in the vortex, repeat the process.

Also, MTT viability was used to characterize the culture: Another 3 samples are placed in a 96-well plate, 100 μL of fresh serum-free fresh culture medium and 10 μL of 12 mM MTT are added to each scaffold and left to incubate for 4 hours at 37° C. After the time period has elapsed, 100 μL of the MTT arrest solution is added to each well and incubated for 4 to 18 hours at 37° C.

Finally, the scaffold pieces are removed from the wells and sized with a ruler; furthermore, the absorbance of each well is measured at a wavelength of 570 nm on the SPECTROstar Omega plate reader.

From the cell count with crystal violet, a growth curve of cells/mL is made.

Subsequently, from the MTT viability assay and the linear fit of the calibration curve for cell viability, the number of viable cells is obtained, which allows the percentage of viability to be obtained using the crystal violet count as the total number of cells:

The following formula was used to calculate viability.

Viability=(Cells determined by MTT·100)/(Cells determined by crystal violet)

Where:

Viability is the percentage of viability.

Cells determined by MTT=Number of cells calculated from the calibration curve with MTT.

Cells determined by crystal violet=Number of cells calculated from a count with crystal violet.

Cell viability is included in the growth curve graph as a secondary axis. If the number of cells determined by MTT is greater than that determined by crystal violet, a viability of 100% can be assumed. If the number of cells determined by MTT is less than 0, a viability of 0% can be assumed.

In addition, an exponential fit to the exponential cell growth section is made to the growth curve, allowing the value of the maximum specific growth rate (μmax) to be obtained as the exponent of the exponential component of the equation. From μmax, the doubling time (td) was calculated as:

t_{d}=(1n(2))/μmax

Where:
t_{d}=Doubling time in hours.
μmax=Maximum specific growth rate in 1/hours.

In contrast, a growth curve of cells/cm′ is made by normalizing the total number of cells counted with crystal violet.

The cells counted with crystal violet were determined by the respective surface area of Durvillaea antarctica scaffolds. The area of the scaffolds can be calculated by assuming that the pieces have a parallelepiped or cube shape, depending on the dimensions of the scaffold.

Finally, a glucose/lactate measurement was made using the following procedure:

Daily, take culture media for each well so as to characterize it. With the culture media taken each day from the cell culture measure glucose and lactate consumption/production as follows: The stored culture media are centrifuged at 1000 RPM (95×g), for 5 minutes and then, a glucose and lactate analysis is performed, using automatic analyzer equipment Y15 and glucose and lactate measurement kits. The results are then recorded in a graph of metabolite concentration vs. hours of culture. Subsequently, the specific rate of metabolite generation or consumption is calculated with equation (5) shown below. Next, the results for the specific rate of glucose consumption and lactate generation are plotted for the time ranges in which the samples were taken. Finally, the glucose/lactate yield is calculated using equation (6) shown below.

$${q}_{met} ( {t}_{i} , {t}_{i - 1} ) = \frac{1}{X ( {t}_{i} , {t}_{i - 1} )} \cdot \frac{{C}_{met} ( {t}_{i} ) - {C}_{met} ( {t}_{i - 1} )}{( {t}_{i} ) - ( {t}_{i - 1} )} \qquad ( 5 )$$

Where:
qₘₑₜ(tᵢ, tᵢ₋₁): Specific rate of metabolite consumption or generation between times tᵢ and tᵢ₋₁, in mM/cell hours.
X(tᵢ, tᵢ₋₁): Average of total cells between times tᵢ y tᵢ₋₁.
Cₘₑₜ(tᵢ): Concentration of the metabolite at time tᵢ in mM.
Cₘₑₜ(tᵢ₋₁): Metabolite concentration at time tᵢ₋₁, in mM.

$${Y}_{lac / glu} = - \frac{{q}_{e , lac}}{{q}_{e , glu}} \qquad ( 6 )$$

Where:
Y_{lac/glu}: Glucose/lactate yield.
q_{e,lac}: Specific rate of lactate generation average of the exponential phase of cell growth.
q_{e,glu}: Specific rate of glucose consumption average of the exponential phase of cell growth.
   Procedure for Suspension Culture on Scaffolds of Durvillaea antarctica

2 spinners of 100 mL, with one magnetic stirrer located 1 mm from the bottom of the vessel, are siliconized with Sigmacote and autoclaved. Repeat the procedure on culturing the scaffolds of Durvillaea antarctica in plates as described above (until the discarding the culture medium by inversion). Dilute the pellet in fresh culture medium such that a concentration of 3.2-10⁶ cells/mL is obtained. To the two spinners add 9.19 mL of fresh culture medium and 5.81 grams of Durvillaea antarctica scaffolds, since this mass of brown algae has a surface area identical to 0.2 grams of microcarrier cytodex 3 (golden standard in microcarriers).

The equivalent concentration of microcarriers is 2.5 g/L. Specifically, in a culture volume of 80 mL, this translates into an adhesion surface area of 400 cm2.

The spinners are inoculated with 5 mL of the cell solution. Then, they are incubated at 37° C. and 5% CO₂ in cycles of 5 minutes of shaking at 35 RPM and 40 minutes of rest for 16 hours, to induce cell adhesion to the Durvillaea antarctica scaffolds.

Ideally, a magnetic stirrer is used with a cycle controller for the above task. Subsequently, the culture medium is discarded and renewed with 74.19 mL of fresh culture medium, leaving it to incubate at 37° C. and 5% CO₂, with a continuous agitation of 35 RPM for 7 days.

Procedure for Characterizing Durvillaea antarctica Suspension Culture on Scaffolds.

Daily, 1 mL of the spinner culture medium is stored to perform a glucose consumption and lactate production curve. From each spinner, a sample of Durvillaea antarctica scaffolds was photographed using procedure previously described above (see the procedure to characterize scaffold culture of Durvillaea antarctica on plate above). Subsequently, 8 pieces of Durvillaea antarctica scaffolds are taken and placed in Eppendorf tubes. 8 scaffolds are generally used, since this is the amount that has the surface area corresponding to 2.5-10⁻³ grams of cytodex 3 microcarriers contained in 1 mL of culture. The scaffolds are washed twice with PBS

Then the Procedure to characterize scaffold culture of Durvillaea antarctica on plate was made as follows: 1 mL of crystal violet is added to each Eppendorf tube and incubated at 37° C. for 1 hr. Subsequently, the samples are vortexed at maximum power for 15 seconds, then a spin is performed in the mini-centrifuge, 10 μL of sample is taken and deposited in a Neubauer chamber and a cell count is performed.

The procedure to characterize scaffold culture of Durvillaea antarctica on plate was made as follows:

From each spinner, 3 samples of Durvillaea antarctica scaffolds with cells are extracted, placed individually in a 96-well plate, 100 μL of fresh serum-free fresh culture medium and 10 μL of 12 mM MTT are added to each scaffold and left to incubate for 4 hours at 37° C. After the time period has elapsed, 100 μL of the MTT arrest solution is added to each well and incubated for 4 to 18 hours at 37° C. Finally, the scaffold pieces are removed from the wells and sized with a ruler. The absorbance of each well is also measured at a wavelength of 570 nm on the SPECTROstar Omega plate reader.

From the cell count with crystal violet, a growth curve of cells/mL is made.

Subsequently, from the viability assay with MTT and the linear adjustment of the calibration curve for cell viability, the amount of viable cells is obtained, which allows one to obtain the viability percentage using the count with crystal violet, as the total cells according to viability equation above.

Additionally, the growth curve is exponentially adjusted to the exponential cell growth section, allowing one to obtain the value of the maximum specific growth rate (μmax), as the exponent of the exponential component of the equation. From μmax, the doubling time (td) was calculated according to the doubling time equation above.

A growth curve of cells/cm2 is made by normalizing the total number of cells counted with crystal violet by the respective surface area of Durvillaea antarctica scaffolds. When extracting 8 pieces of scaffolds, which are equivalent to a surface area of 2.5-10⁻³ grams of cytodex 3 microcarriers, the surface area should be 5 cm2.

Finally, the glucose-lactato were analyzed using the above procedure to characterize scaffold culture of Durvillaea antarctica on plate as follows: The stored culture media are centrifuged at 1000 RPM (95×g), for 5 minutes and then, glucose and lactate analysis are performed, using the automatic analyzer equipment in Random Access Y15 and the glucose and lactate measurement kits. The results are then recorded on a graph of metabolite concentration vs. hours of culture. Subsequently, the specific rate of metabolite generation or consumption is calculated with equation (5) above. Next, the results for the specific rate of glucose consumption and lactate generation are plotted for the time ranges in which the samples were taken. Finally, the glucose/lactate yield is calculated with above equation (6).

Procedure for Performing Differentiation of C2C12 Cells on Durvillaea antarctica Scaffolds in Suspension

From a suspension culture of C2C12 cells on Durvillaea antarctica scaffolds at their highest cell concentration, all the culture medium is removed. If the culture is grown in 80 mL, scaffold surface area of 400 cm2 and an initial seeding of 1-10⁵ cells/mL or a ratio of these values, then the maximum concentration will be reached at 80 hours. 80 mL of DMEM/F12 2% HS differentiation medium is added. The spinners are incubated at 37° C., 5% CO₂ with continuous 35 RPM agitation. Every 48 hours, the differentiation medium is discarded and renewed with 80 mL of fresh differentiation medium. The culture should be extended for at least 4 days and a maximum of 6 days.

Decellularization of Durvillaea antarctica (Cochayuyo)

The above procedures and the following examples are described now with reference to the figures.

Cochayuyo was decellularized using the protocol described herein and experiments were made in triplicate using as starting material a volume of 15 ml of dried cochayuyo cortex. The decellularization process was measured quantifying the DNA content of the supernatant using nanodrop equipment. Also, the decellularization was photographed at each stage of the process. After neutralization, the DNA content within decellularized vs. cellularized algae was compared by DNA extraction with liquid nitrogen and CTAB buffer, and then measured in nanodrop. The significant difference was analyzed with t-student in Graphpad Prism 8.

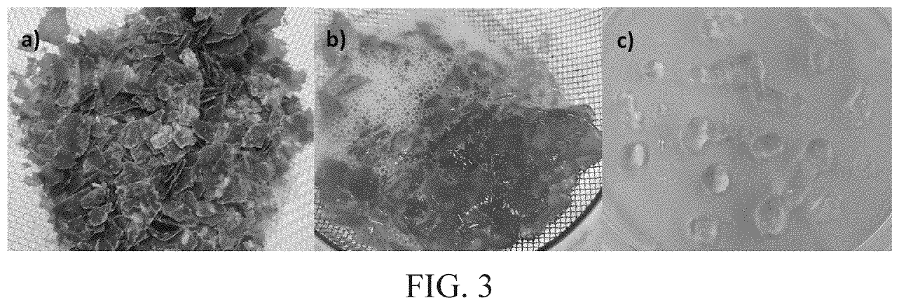

FIG. 3 shows the staining of Durvillaea antarctica during scaffold manufacture with a) representing Crushed cochayuyo. b) representing Cochayuyo after 5 days of decellularization with SDS, and c) representing Cochayuyo after neutralization process. In FIG. 3, it is possible to see an evident color change in the scaffold. Specifically, at the end of neutralization, the scaffolds are transparent.

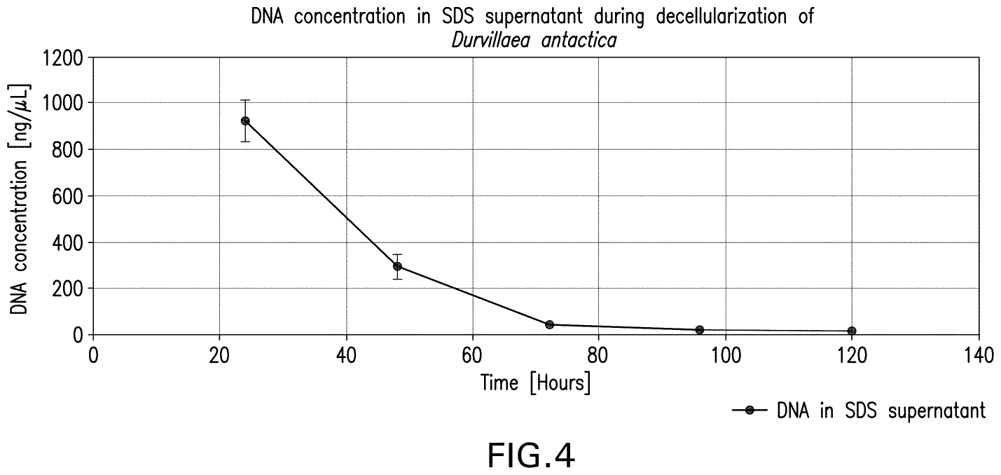

Subsequently, the remaining DNA in the supernatant was measured during decellularization. The DNA should be reduced during decellularization, indicating successful decellularization due to the cell content being removed from the cortex. FIG. 4 shows the DNA concentration in the SDS supernatant during the decellularization period of Durvillaea antarctica. In FIG. 4, it is possible to see that DNA was removed by SDS, and the removal of DNA from the algae decreases over time, indicating that the cellularization process is complete. After the third day, the change is negligible, so decellularization could be shortened to 3 days.

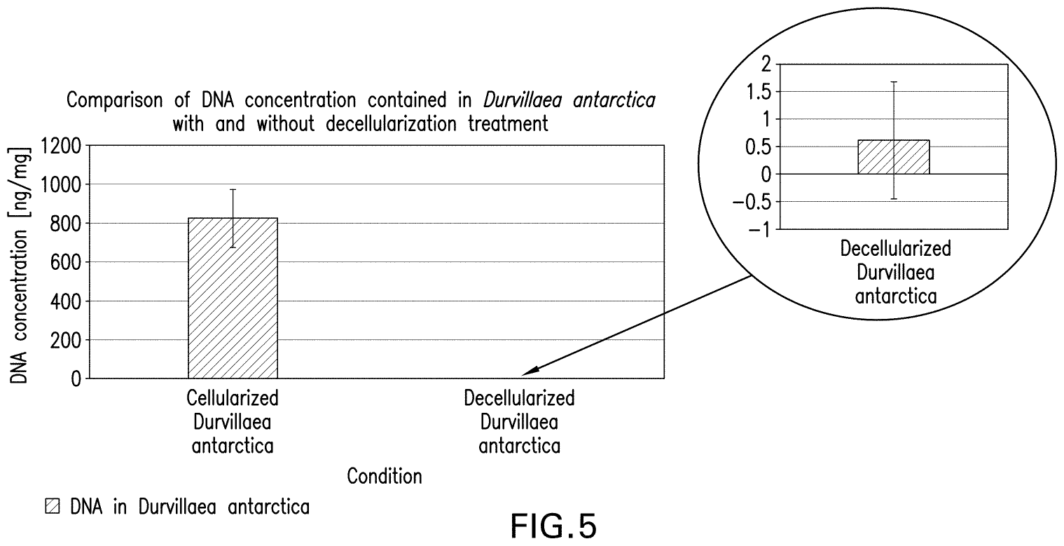

FIG. 5 shows the concentration of genetic material in Durvillaea antarctica before and after the complete process. In FIG. 5, DNA quantification was observed at the beginning of the procedure and the end. There is a significant difference between the DNA content and the procedure shows the effective removal of the DNA from the algae.

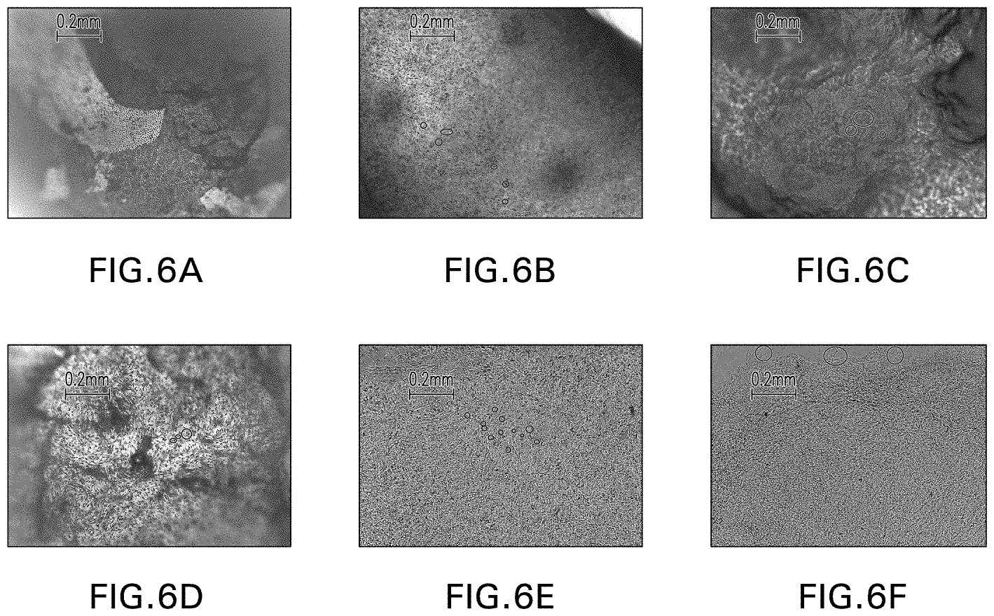

FIG. 6 shows Top row brown algal structure, bottom row decellularization level of Durvillaea antarctica (compression with coverslip). a) and d) cellularized cochayuyo (untreated)×10. b) and e) 5 days of decellularization with SDS×10. c and f) After neutralization process ×10. Cells are enclosed in a red circle, while hollows are shown in yellow circles. In FIG. 6, it was determined that the cochayuyo was decellularized, because at the end of the process, no cells were observed.

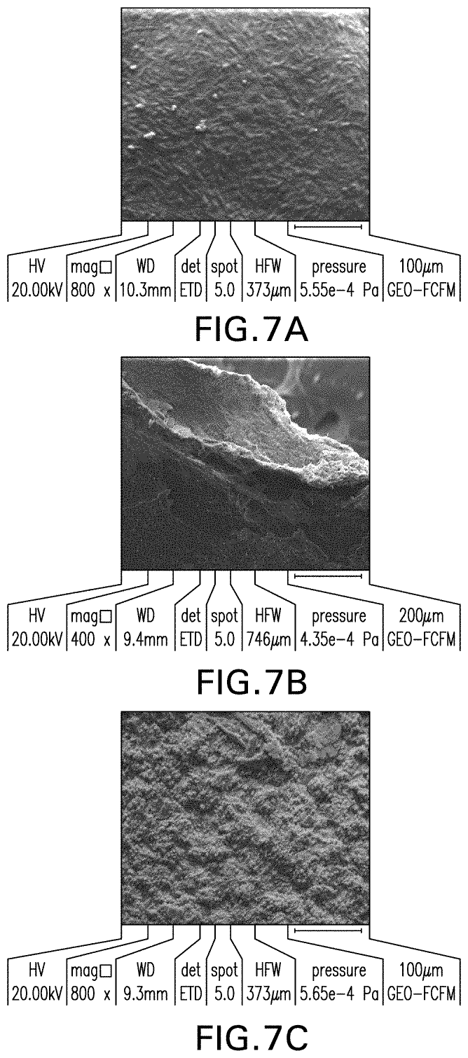

FIG. 7 shows SEM images (FEI Quanta 250) of the structure of dried Durvillaea antarctica with a) representing Cellularized cochayuyo (without treatment), and b) and c) representing Cochayuyo after the decellularization process with SDS. FIG. 7 shows that a modification of the algal surface is observed after the decellularization process with SDS.

### C2C12 Culture in Adhesion on Plates

The culture was made according to the protocol disclosed herein. For these assays, C2C12 cells were used with mouse cell line myoblast progenitor. The protocols disclosed herein can be used in other cell types, and are relevant for cultivated meat.

Plate cultures were used in triplicate. Suspension culture was in duplicate, and the culture was grown for 7 days with sampling every 24 hours, in a culture volume of 80 ml.

A C2C12 differentiation protocol was used comprising 4 days of growth and 6 days of differentiation, with sampling every 48 hours during differentiation, in a culture volume of 120 mL. The protocol used was as discussed herein.

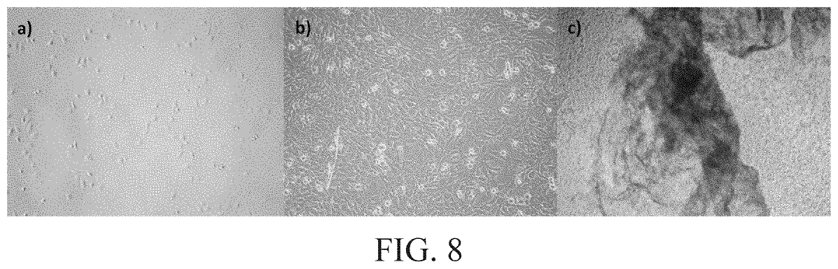

FIG. 8 shows the plate culture of C2C12 at a) 2 hours of ×10 culture, and at b) 26 hours of ×10 culture, and at c) 100 hours of culture ×10 (with augmentation). FIG. 8 shows the C2C12 adherent culture on plates and the differentiation of these cells into muscle. Their ability to differentiate was observed and measured. Cell growth is evident after 26 hours of culture. After 100 hours of culture, cell fusion is generated because of cell differentiation, which causes a contraction that detaches the cells from the plate.

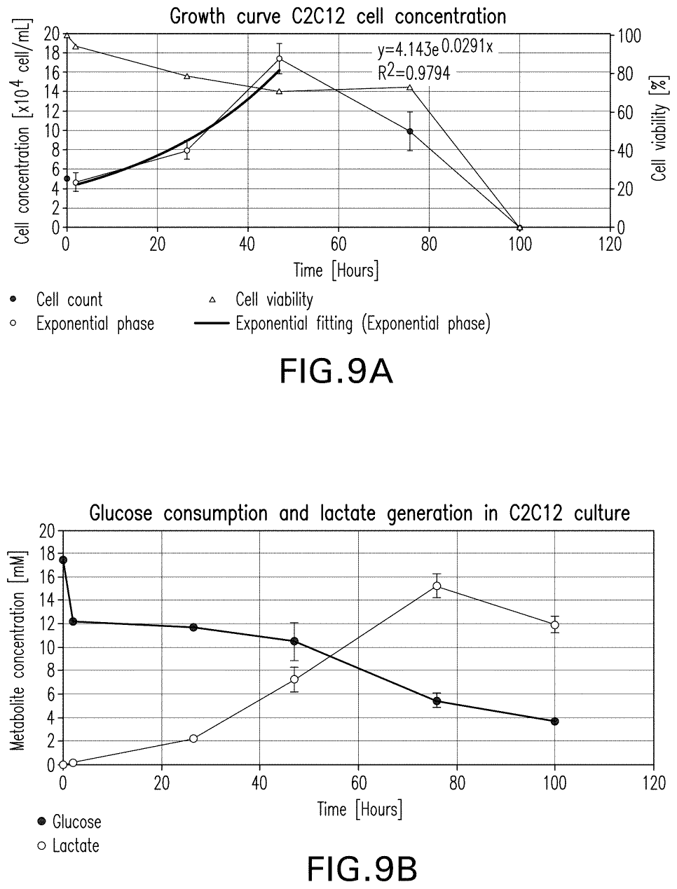

FIG. 9 shows the plate culture of C2C12 with a) representing the growth curve and b) representing the consumption and generation of metabolites. Cell growth and glucose-lactate consumption were measured, and the results are shown in FIG. 9. In FIG. 9, it was determined that the exponential phase is 48 hours, Viability is maintained at about 70% until 72 hours, and Glucose is not completely depleted. With these results, the scaffolding relative to the control conditions were compared.

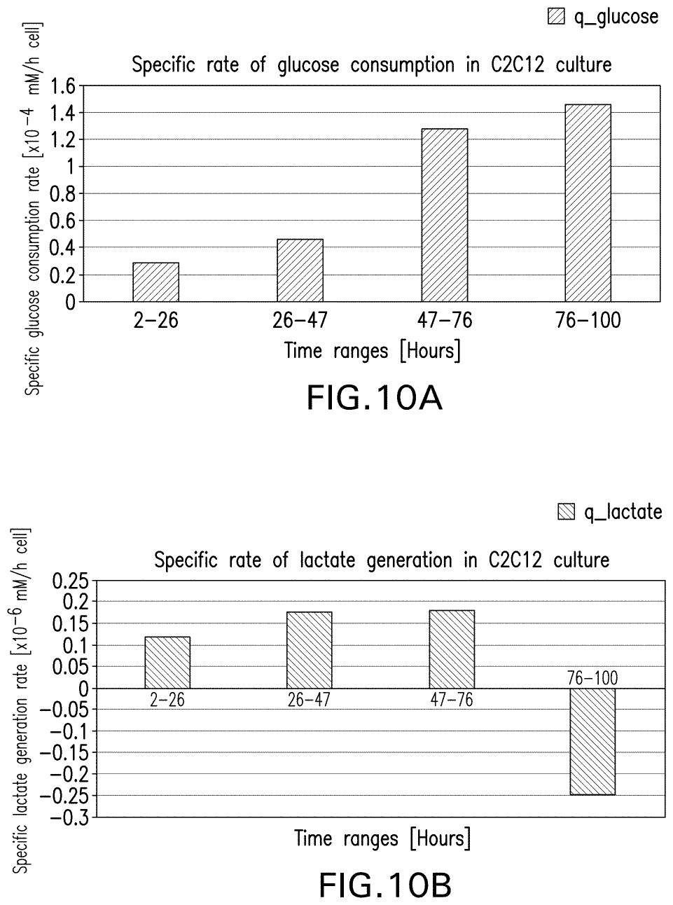

FIG. 10 shows the specific rates of a) glucose consumption and b) lactate generation culture of C2C12 in plates. In FIG. 10 shows the lactate and glucose-specific rates show that a decrease in lactate is generated between 76 and 100 hours, so it is determined that this metabolite is consumed. Moreover, there is greater glucose consumption during the last period of the culture.

Table 1 shows a comparison of the culture parameters in plates at 37° C. and 5% CO₂ (VALAVANIS, C, 2021) (MONTESANO, A, 2015) with the experimental data obtained herein. Those results will be helpful for the analysis of the scaffolding's performance of the present invention.

TABLE 1

| | Experimentally | | Bibliography | |
|---|---|---|---|---|
| Parameter | determined value | | value | |
| μₘₐₓ | 2.91 · 10⁻² | [1/h] | 4.33 · 10⁻² | [1/h] |
| t_{d} | 23.82 | [h] | 16 | [h] |
| Max concentration | 17.39 | [×10⁴ cell/mL] | 25 | [×10⁴ cell/mL] |

| | | |
|---|---|---|
| Y_{lac/glu} | 0.39 | — |

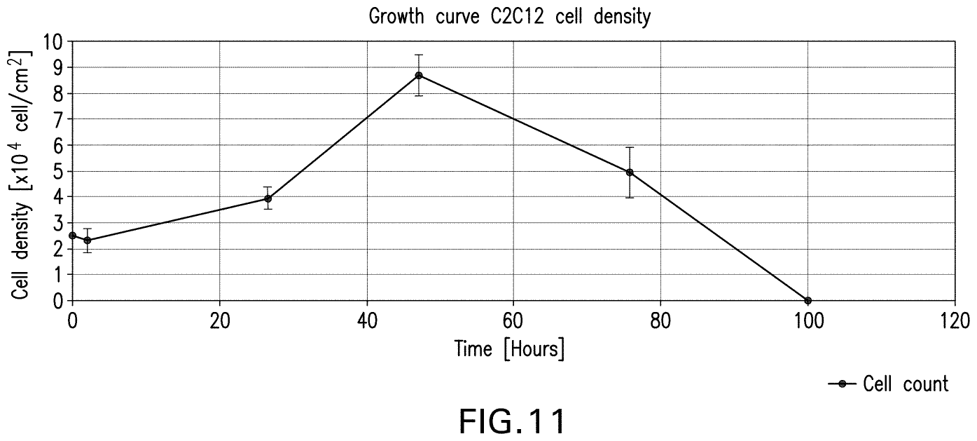

The cell density of the culture was measured and is shown in FIG. 11. FIG. 11 shows cellular density of C2C12 on plates at different times of culture. This provides the data to compare with the scaffolding of the present invention.

### C2C12 Culture on Cochayuyo Scaffoldings

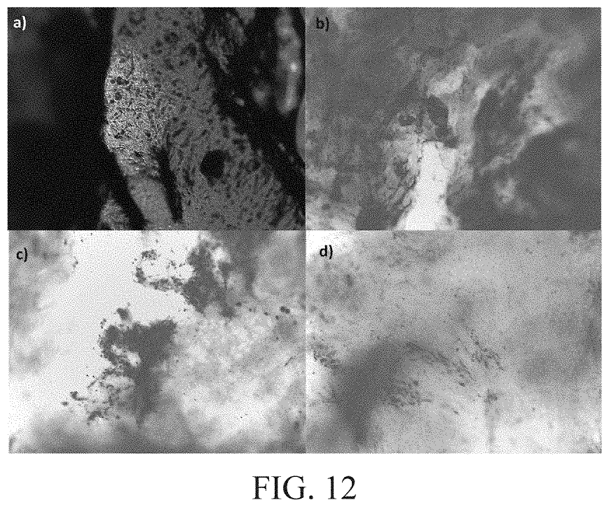

Subsequently, C2C12 cells were grown on cochayuyo scaffoldings, and the results are shown in FIG. 12. FIG. 12 shows C2C12 culture on Durvillaea antarctica scaffolds on plates. a) 2 hours of ×10 culture. b) 73 hours of ×10 culture. c) 122 hours of ×10 culture. d) 168 hours of ×10 culture ×10 culture. After 2 hours, cell adhesion is observed due to the shape of the cells. At 73 hours, cell growth is evident. From 122 to 168, there is cell death.

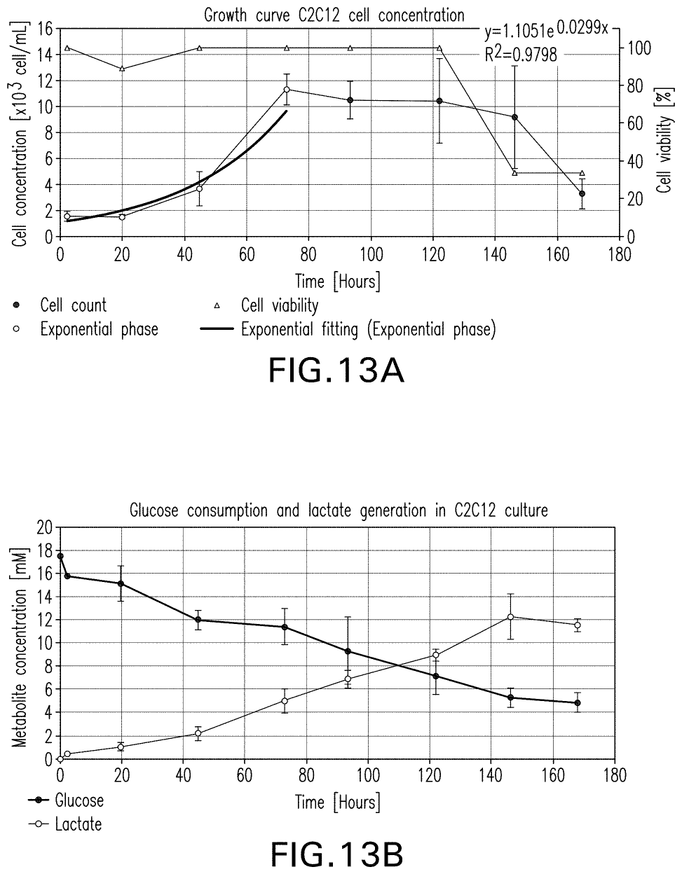

FIG. 13 shows C2C12 culture on Durvillaea antarctica scaffolds on plates. a) Growth curve. b) Consumption and generation of the metabolites glucose/lactate. The exponential phase is observable at 72 hours. Viability remains close to 100% until 120 hours. Glucose is not completely depleted.

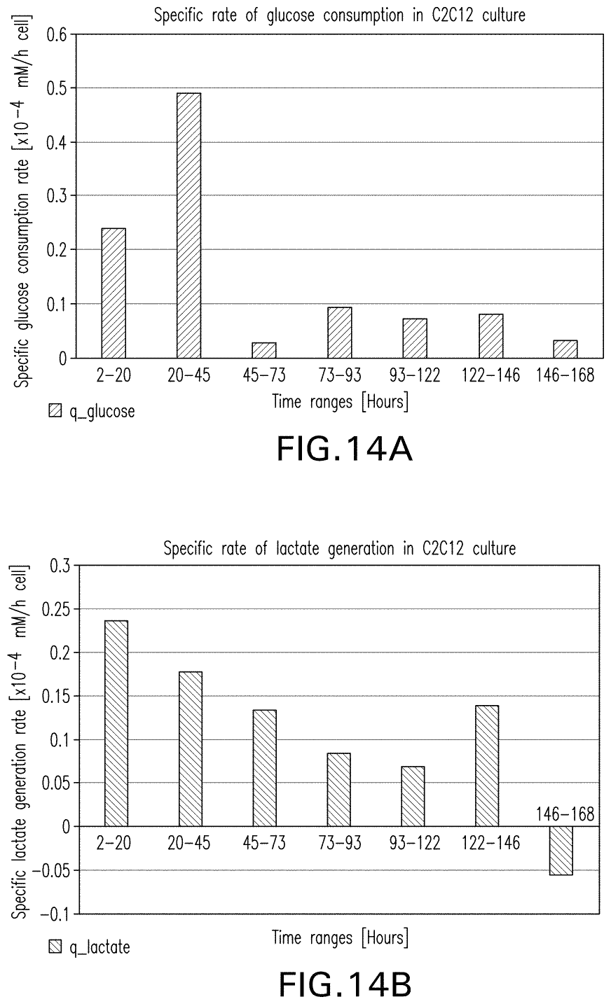

FIG. 14 shows the specific rates of metabolite generation or consumption, C2C12 culture on Durvillaea antarctica scaffolds on plates with a) representing glucose consumption and b) representing lactate generation. There is a decrease in lactate, generated between 146 and 168 hours, so it was determined that this metabolite is consumed. There appears to be higher glucose consumption during the first period of the culture.

Table 2 shows the comparison of parameters of C2C12 culture on Durvillaea antarctica scaffolds on plates at 37° C. and 5% CO₂. These results provide useful data for the analysis of the performance of the scaffolding of the present invention.

TABLE 2
Parameters of C2C12 culture on Durvillaea antarctica
scaffolds in a plate at 37° C. and 5% CO2.

| Parameter | Experimentally determined value | |
|---|---|---|
| μₘₐₓ | 2.99 · 10⁻² | [1/h] |
| t_{d} | 23.18 | [h] |
| Max concentration | 11.33 | [×10³ cell/mL] |

| | |
|---|---|
| Y_{lac/glu} | 0.72 |

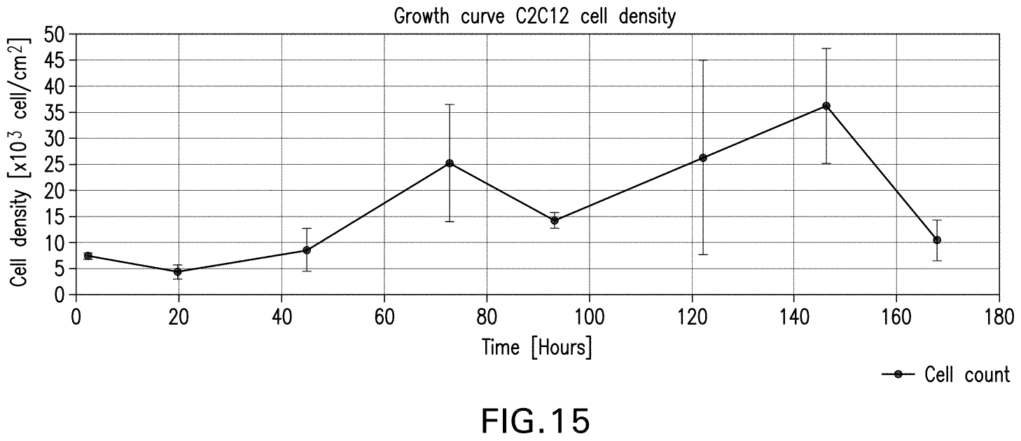

The cellular density measures how many cells are using the surface of the scaffolding of the present invention. FIG. 15 shows the cell density of C2C12 culture on Durvillaea antarctica scaffolds on a plate. A significant error bar is seen due to the difference in cell adhesion. The number of cells adhering depended on the dimensions of the scaffold. In the absence of other instances for adhesion, the larger scaffolds adhered to many cells relative to the smaller scaffolds.

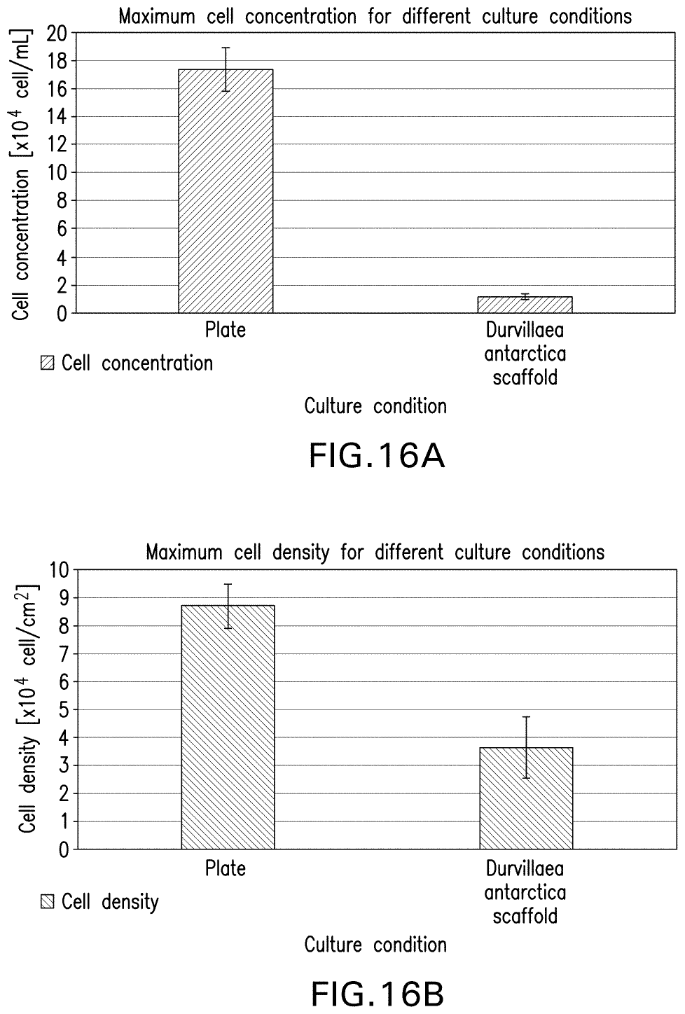

FIG. 16 shows a comparison of the maximum biomass concentration achieved in plate and scaffold cultures of Durvillaea antarctica with a) showing results of cell concentration p<0.05, and b) showing results of cell density p<0.05.. A significant difference was seen in concentration since the scaffolds use a small portion of the plate area. In terms of cell density, due to the lack of agitation, the cells only adhered in the focused sectors, while in the plate, they used the entire surface.

### Cell Culture on Suspension

C2C12 cells are not able to grow without attachment. Therefore, they need a surface to grow. To overcome this lack of attachment, the use of microcarriers, spherical beads with adhesion capabilities can be used. Moreover, collagen-covered microcarriers have excellent performance and are commonly used (such as a Cytodex 3 (Sigma-Aldrich)). The cell culture was made as described, and the same surface as described was used for the microcarriers and seaweed scaffoldings. At 16 h of culture, there is a media change and specific metabolites rates can change. This media change is for all the cultures.

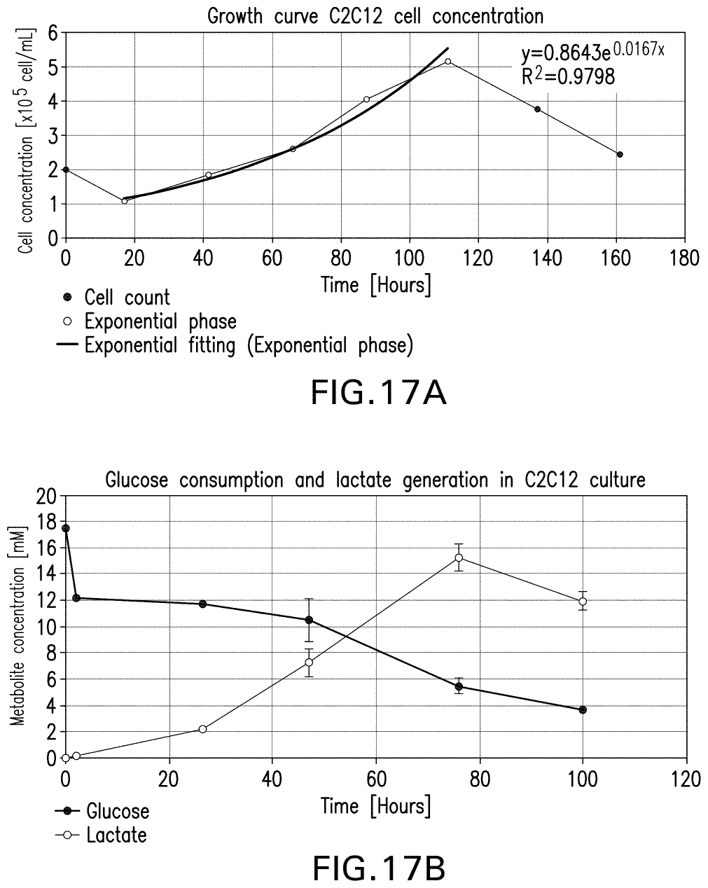

FIG. 17 shows C2C12 culture using microcarriers with a) representing a growth curve and b) representing consumption and generation of metabolites. FIG. 17 shows a growth curve where the exponential phase is seen at 110 hours, and growth is more prolonged than in cochayuyo scaffolds measured on plates. Also, FIG. 17 shows the results of metabolite production-consumption where glucose does not run out entirely, and cell death coincides with a higher concentration of lactate than with glucose.

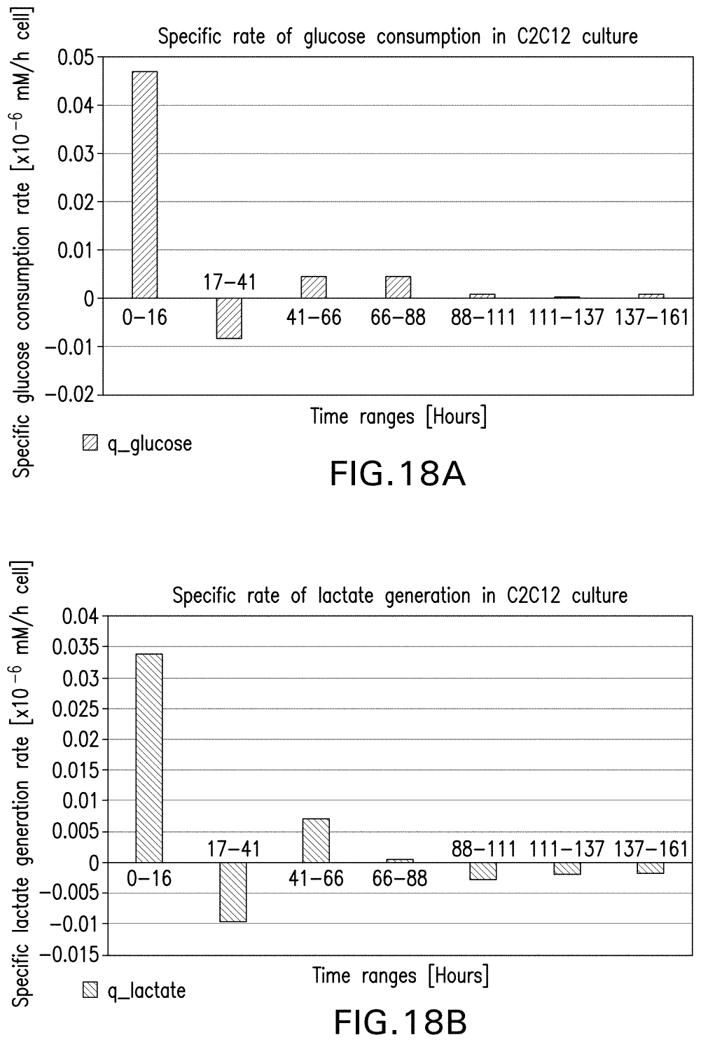

FIG. 18 shows the specific rates of metabolite generation or consumption of a C2C12 culture in microcarriers with a) representing glucose consumption and b) representing lactate generation. It can be seen that at the beginning, there is a high consumption since the adhesion is done in a low volume of medium. After the change of medium, consumption rates are not very high.

Table 3 shows the experimental results of C2C12 culture parameters compared with a reference example.

TABLE 3
Parameters of C2C12 culture in microcarriers at 37°
C., 35 RPM and 5% CO2 (BREESE, T. W, 1999) (RISNER, D, 2021).

| | Experimentally | | Bibliography | |
|---|---|---|---|---|
| Parameter | determined value | | value | |
| μₘₐₓ | 1.67 · 10⁻² | [1/h] | 1.92 · 10⁻² | [1/h] |
| t_{d} | 41.50 | [h] | 36 | [h] |
| Max concentration | 5.15 | [×10⁵ cell/mL] | 90 | [×10⁵ cell/mL] |

| | | |
|---|---|---|
| Y_{lac/glu} | 0.49 | — |

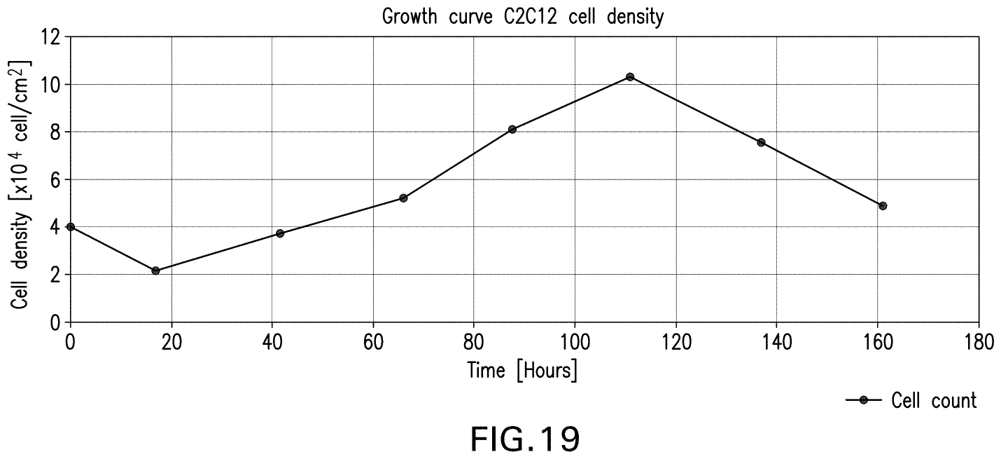

FIG. 19 shows the cell density of C2C12 culture in microcarriers.

### C2C12 Culture on Cochayuyo Scaffoldings as a Microcarriers in Suspension Cultures

Subsequently, C2C12 cultures were made on cochayuyo scaffoldings in suspension cultures. In that type of culture, the scaffoldings of the present invention behave as microcarriers and confer a good scalability quality. The experiment was made using the same protocol as described above with the microcarriers.

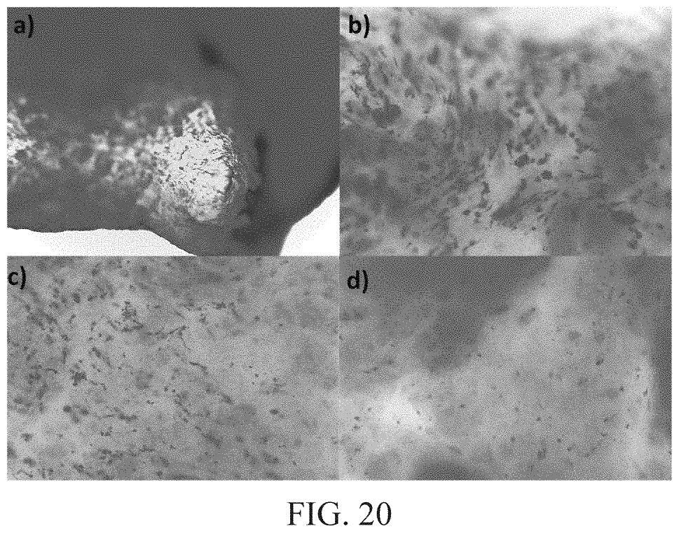

FIG. 20 shows the culture of C2C12 on Durvillaea antarctica scaffolds in suspension with a) showing 16 hours of ×10 culture, b) showing 70 hours of ×10 culture, c) showing 116 hours of ×10 culture, and d) showing 164 hours of ×10 culture. In FIG. 20, after 16 hours, cell adhesion is observed which is evidenced due to the shape of the cells, then at 70 and 116 hours, cell growth is evidenced, and at 164 hours, cell death is observed.

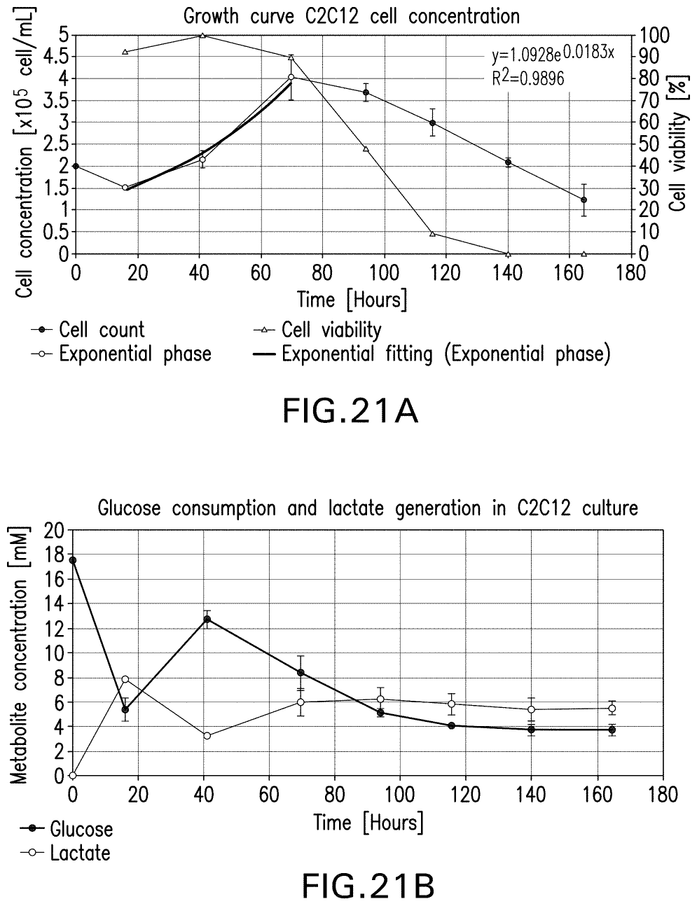

FIG. 21 shows C2C12 culture on Durvillaea antarctica scaffolds in suspension with a) showing a growth curve and b) showing consumption and generation of metabolites. In FIG. 21, it is possible to see the cell growth, where the exponential phase is 72 hours. It is apparent from FIG. 21 that viability remains close to 100% until 120 hours and Glucose is not completely depleted.

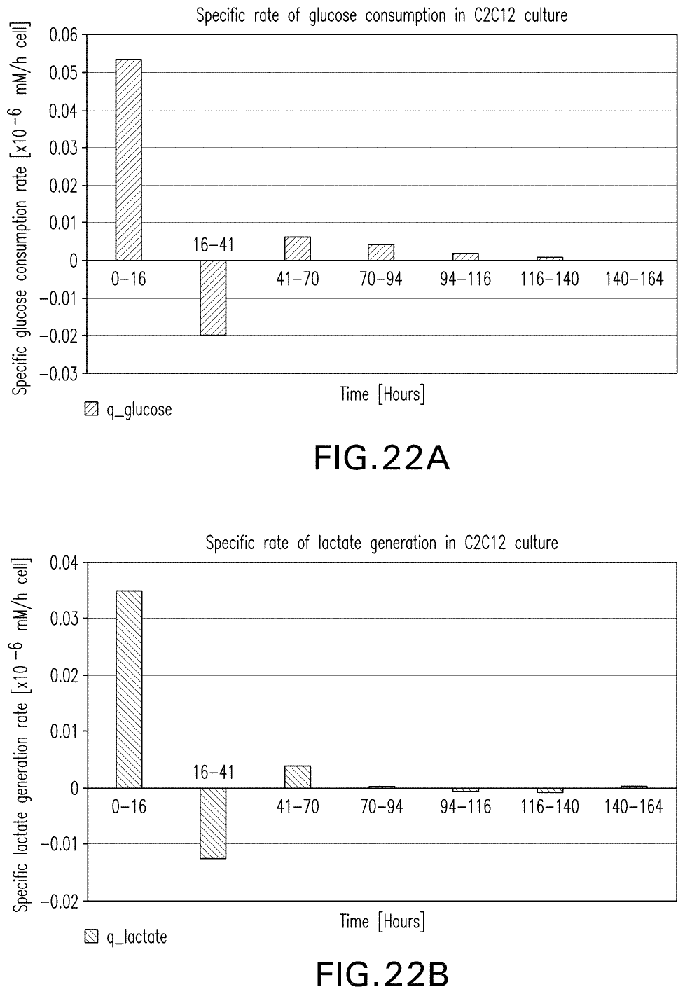

FIG. 22 shows the specific rates of metabolite generation or consumption, in a C2C12 culture on suspended Durvillaea antarctica scaffolds in suspension with a) representing glucose consumption and b) representing lactate generation. At the beginning, there is a high consumption since the adhesion is done in a low volume of medium. After the change of medium, consumption rates are not very high.

Table 4 shows the experimental parameters of cell culture on the scaffoldings of the present invention in suspension culture.

TABLE 4
Parameters of C2C12 culture on Durvillaea antarctica
scaffolds in suspension at 37° C., 35 RPM and 5% CO₂.

| Parameter | Experimentally determined value | |
|---|---|---|
| μₘₐₓ | 1.83 · 10⁻² | [1/h] |
| t_{d} | 37.88 | [h] |
| Max concentration | 4.03 | [×10⁵ cell/mL] |

| | |
|---|---|
| Y_{lac/glu} | 0.62 |

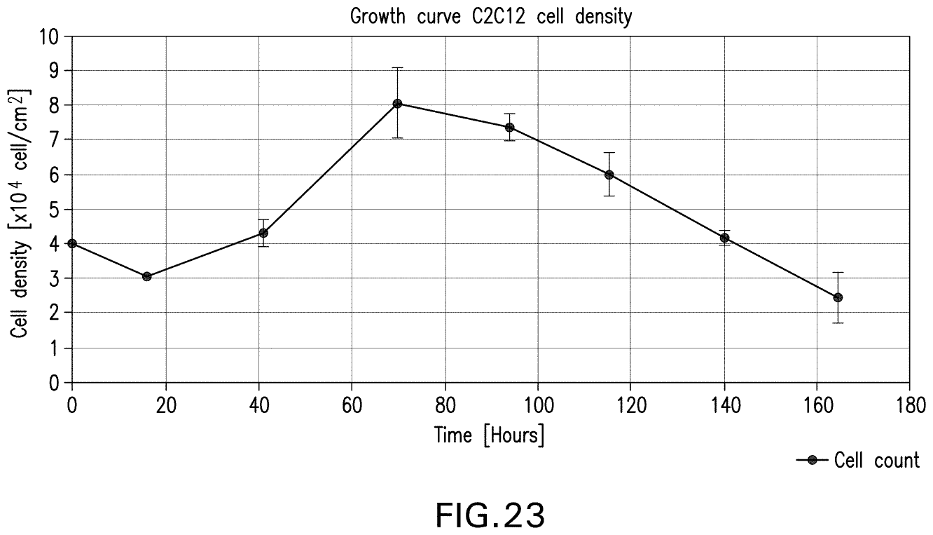

FIG. 23 shows the cell density of C2C12 culture on Durvillaea antarctica scaffolds in suspension.

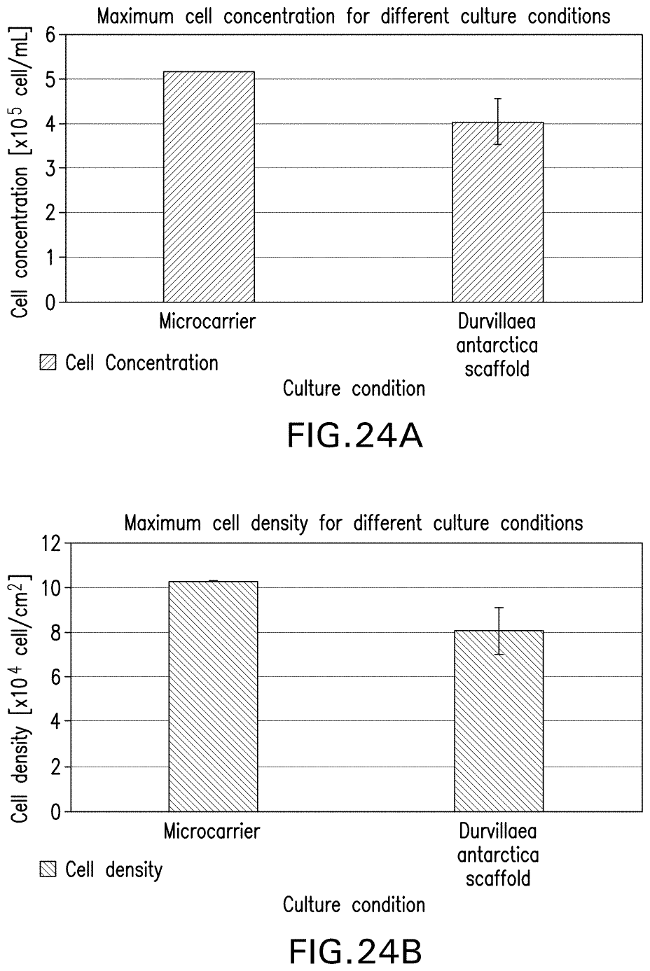

FIG. 24 shows a comparison of maximum biomass concentration achieved in microcarrier and scaffold cultures of Durvillaea antarctica in suspension with a) representing the cell concentration, t-student P<0.05. FIG. 24 shows that there is no significant difference in cell density. However, a lower cell concentration and density is observed since the cells do not use 100% of the scaffold surface.

In plate cultures, the cochayuyo scaffolds grew at a similar rate but it presents a lower cell concentration. The cochayuyo scaffolds have a higher glucose/lactate yield, but in both conditions, it is between 0 and 1.5, so they are considered efficient. In suspension cultures, the cochayuyo scaffold is faster for cell growth. Although it has a lower cell concentration, its density does not significantly differ. In addition, the cochayuyo scaffolds have a higher glucose/lactate yield, but in both conditions, it is between 0 and 1.5, so they are considered efficient. This means that in suspension cultures cochayuyo scaffolds can produce equivalent cell yield in less time.

Table 5 shows a summary of experimental parameters for the different growing conditions.

TABLE 5
Summary of experimental parameters for the different growing conditions.

| | Experimentally determinded value | | | | | | | |
|---|---|---|---|---|---|---|---|---|
| | | | Durvillaea antactica | | | | Durvillaea antactica | |
| | | | scaffold | | | | scaffold | |
| Parameter | Plate | | in plate | | Microcarriers | | in suspension | |
| μₘₐₓ | 2.91 · 10⁻² | [1/h] | 2.99 · 10⁻² | [1/h] | 1.67 · 10⁻² | [1/h] | 1.83 · 10⁻² | [1/h] |
| t_{d} | 23.82 | [h] | 23.18 | [h] | 41.50 | [h] | 37.88 | [h] |
| Max concentration | 17. [symbol] 9 | [×10 [symbol] cell/mL] | 1.13 | [×10 [symbol] cell/mL] | 51.50 | [×10 [symbol] cell/mL] | 40. [symbol] | [×10 [symbol] cell/mL] |

| | | | | |
|---|---|---|---|---|
| Y_{lac/glu} | 0.36 | 0.72 | 0.49 | 0.62 |

[symbol]

indicates data missing or illegible when filed

### Ulva Decellularization

Ulva was decellularized due to its use as a scaffold using a similar protocol from a reference. Subsequently, tests were performed with this alga to compare it with the scaffolds of the present invention. Afterward, Ulva was decellularized using the protocol “Scaffolding-microcarrier generation.” Due to the mass required to perform the suspension culture and store stock, 70 g of Ulva in a 2-liter flask was used. During decellularization with 1% SDS, the DNA content of the supernatant was measured using nanodrop equipment. The decellularization was photographed at each stage of the process. After neutralization, the DNA content of decellularized vs. cellularized algae was compared by DNA extraction with liquid nitrogen and CTAB buffer, and then the samples were measured in nanodrop. Significant differences were seen using t-student in Graphpad Prism 8.

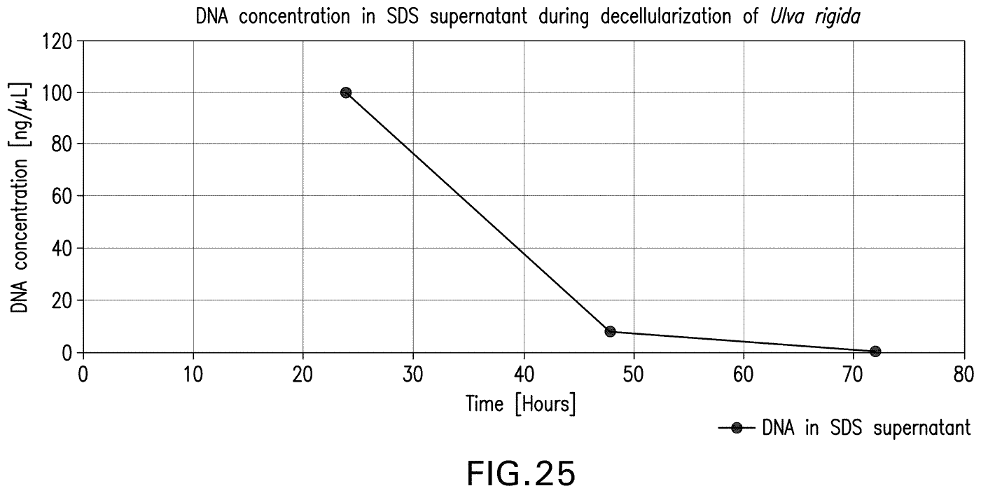

FIG. 25 shows DNA contained in the SDS supernatant during the decellularization period of Ulva. FIG. 25 shows, unlike the cochayuyo, after 3 days of decellularization with 1% SDS, there is no evidence of DNA removal, so the process was not continued.

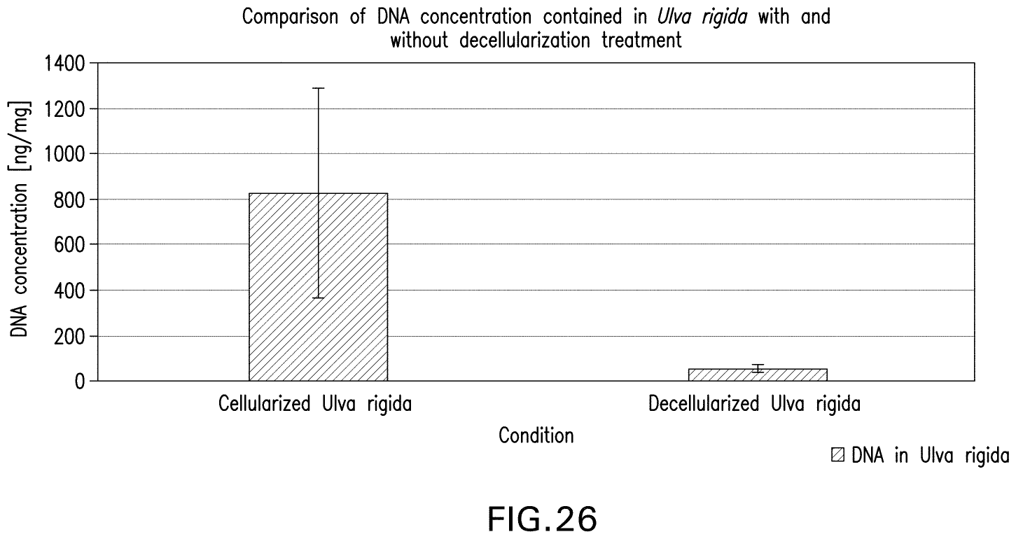

FIG. 26 shows DNA Concentration in Ulva rigida before and after the complete process with *P<0.05. FIG. 26 shows that there is a significant difference in DNA content, so the procedure was effective. Decellularized Ulva has a DNA concentration of 58.9 [ng/mg], so it was not considered to be decellularized, with a number that is close to the limit established by the literature of 50 [ng/mg] (Gershlak, 2017).

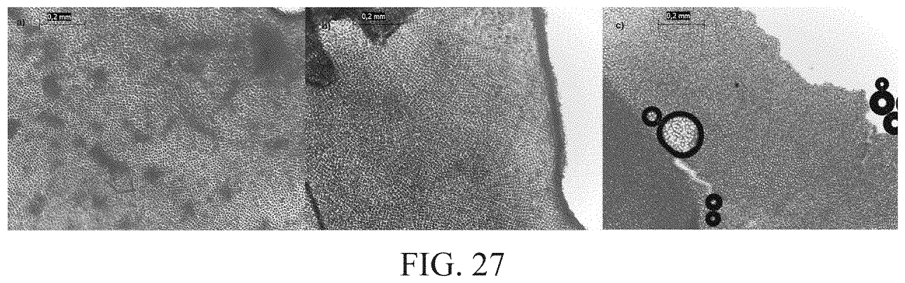

FIG. 27 shows photographs of Ulva at three different stages of decellularization with a) showing cellularized Ulva, staining with crystal violet ×10, b) showing Ulva after 3 days of decellularization with SDS 1%, staining with crystal violet ×10, and c) showing Ulva after neutralization, staining with crystal violet ×10. In the first image, the nuclei stained black can be observed. After decellularization with SDS, a smaller number of nuclei is observed. Finally, after neutralization, the algal structure without nuclei is observed. It was determined that the procedure was effective in removing the Ulva cells.

### C2C12 Culture on Ulva Scaffoldings

Suspension cultures work better with algae scaffoldings. Accordingly, a suspension culture of C2C12 cells was made using Ulva scaffoldings with the same parameters used for cochayuyo. After 7 hours, cell adhesion was observed, as evidenced by the shape of the cells. No growth is evident with the naked eye between 7 and 71 hours. At 120 hours, cell death is observed.

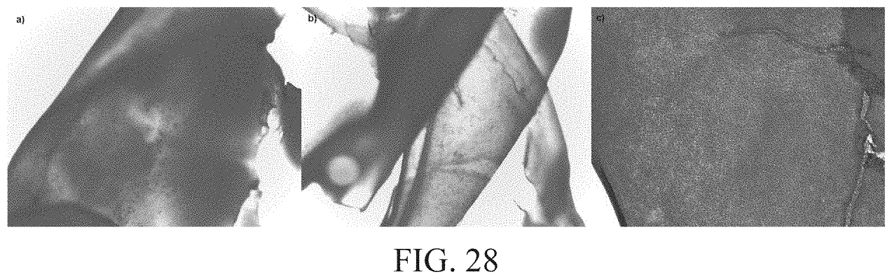

FIG. 28 shows the C2C12 culture on Ulva rigida scaffolds in suspension with a) showing at 7 hours of culture ×10 b) showing the results after 71 hours of culture ×10, and c) showing the results after 120 hours of culture ×10.

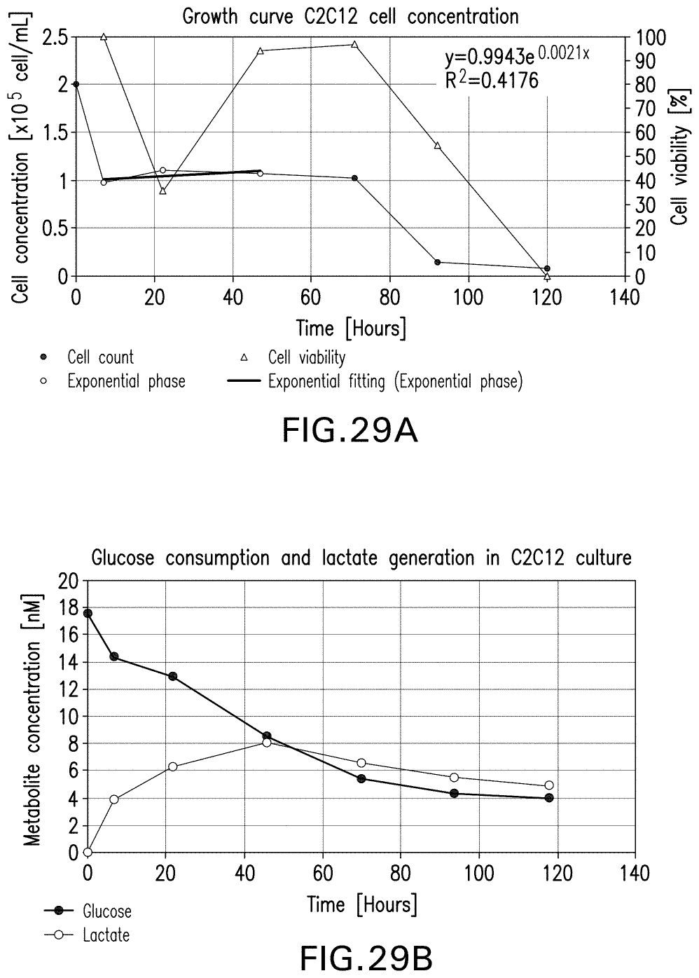

FIG. 29 shows the results of a culture of C2C12 on Ulva rigida scaffolds in suspension with a) representing the growth curve and b) representing the consumption and generation of metabolites. FIG. 29 shows the cells do not appear to undergo exponential growth. Viability remains near 100% until 80 hours, then declines dramatically. Glucose is not entirely depleted, and the sample seems to consume lactate.

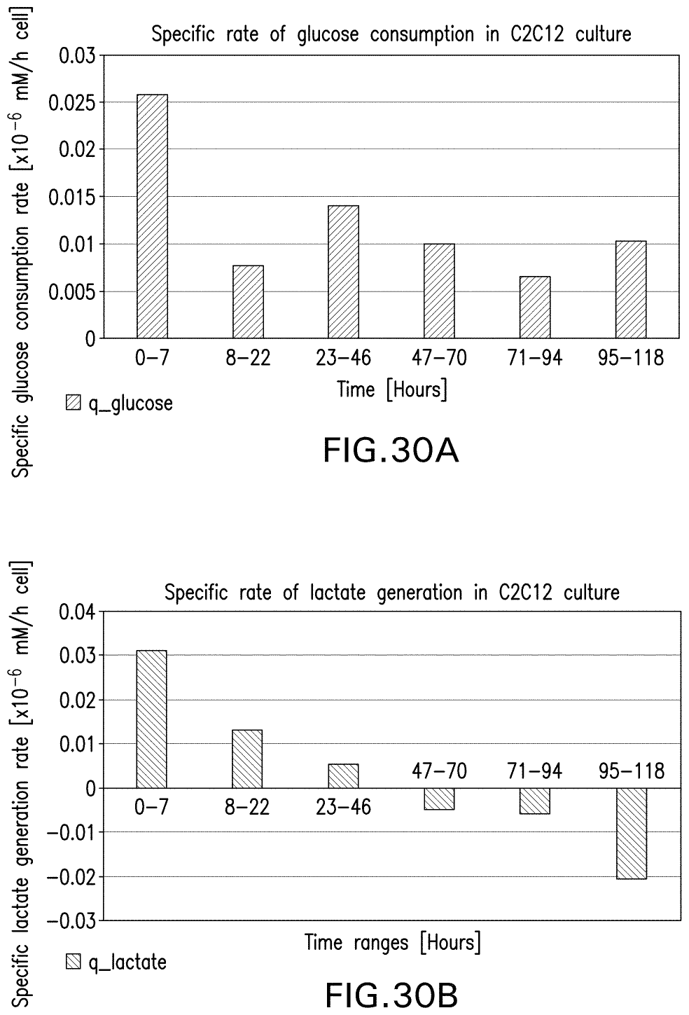

FIG. 30 shows specific metabolite generation or consumption rates in C2C12 culture on Ulva rigida scaffolds in suspension with a) representing glucose consumption and b) representing lactate generation. FIG. 30 shows lactate consumption and glucose is not entirely consumed.

Table 6 shows the experimental parameters of C2C12 culture on Ulva rigida scaffolding.

TABLE 6
Parameters of C2C12 culture on Ulva rigida scaffolds
in suspension at 37° C., 35 RPM and 5% CO2.

| Parameter | Experimentally determined value | |
|---|---|---|
| μₘₐₓ | 2.10 · 10⁻³ | [1/h] |
| t_{d} | 330.07 | [h] |
| Max concentration | 1.10 | [×10⁵ cell/mL] |

| | |
|---|---|
| Y_{lac/glu} | 0.17 |

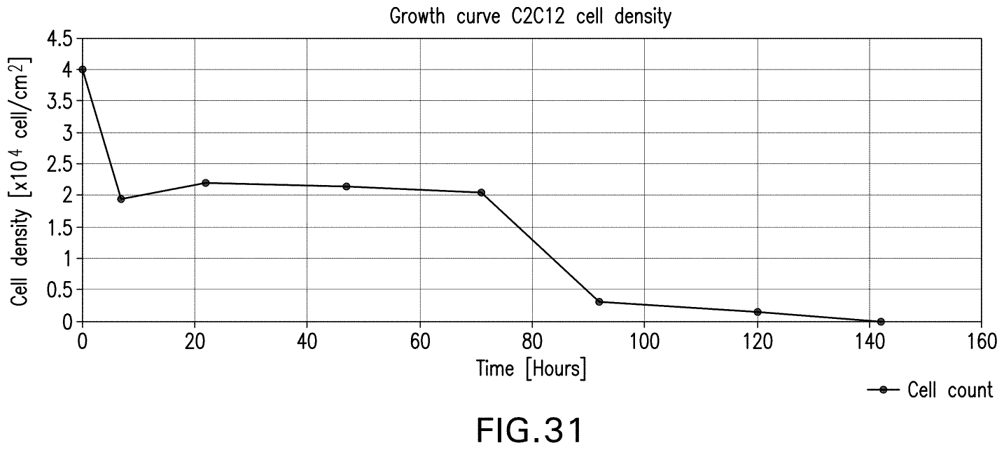

FIG. 31 shows the cell density of C2C12 culture on Ulva rigida scaffolds in suspension.

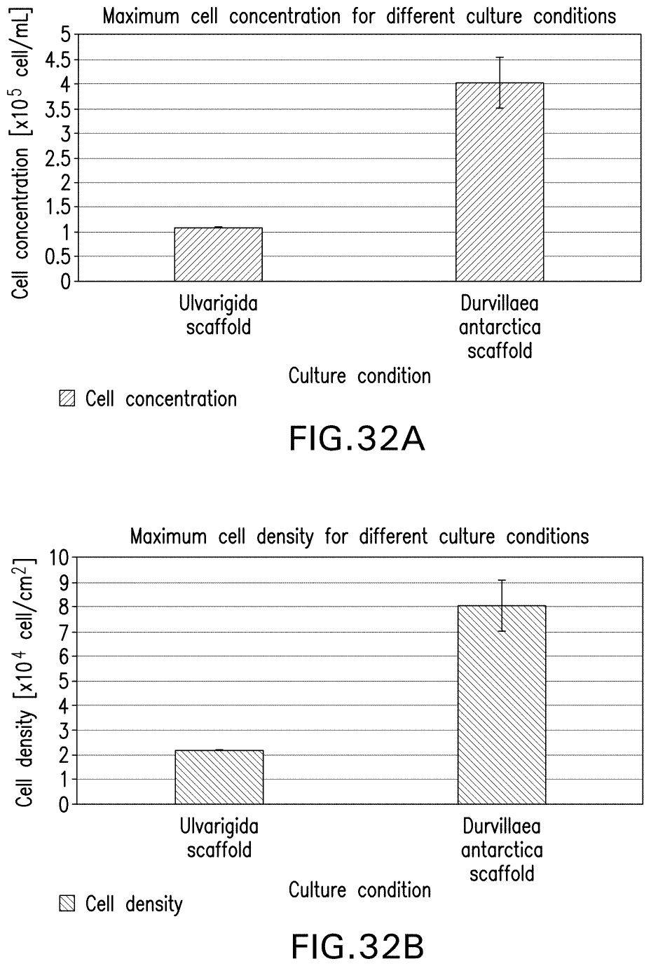

Comparing suspension cultures using Cochayuyo and Ulva, it is clear that there are significant differences in cell concentration and cell density, where cochayuyo has significantly better performance with comparable surface areas under cultivation (see FIG. 32). FIG. 32: shows a comparison of maximum biomass concentration achieved in cultures on Ulva rigida scaffolds and on Durvillaea antarctica scaffolds in suspension with a) showing cell concentration P<0.05 and b) showing Cell density P<0.05.

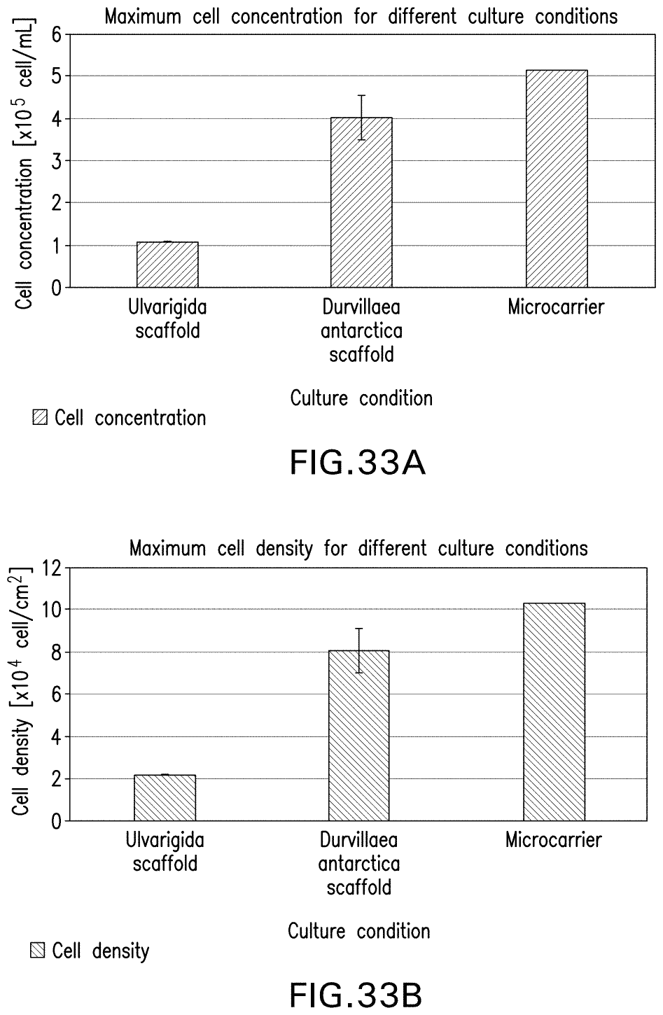

Finally, suspension cultures were compared between Ulva, Durvillaea, and microcarriers (cytodex 3). FIG. 33 shows a comparison of maximum biomass concentration achieved in cultures on Ulva rigida scaffolds, on Durvillaea antarctica scaffolds and microcarriers in suspension with a) representing cell concentration, and b) representing cell density. Ulva has a lower performance in cell concentration and cell density, and Durvillaea's performance is comparable when used with a microcarrier, mostly in cell density. There is no significant difference between the two samples in cell density, even though cochayuyo has a lower cell concentration and the exponential time of the cells is also lower also (see FIG. 33).

It should be appreciated that the following methodologies disclosed in the following references have been incorporated by reference in their entireties: J. D. Jones, et al., “Decellularized Spinach: an Edible

Scaffold for Laboratory-Grown Meat,” Food Bioscience, 2021, Vol. 41, 100986; J. R. Gershlak, et al., “Crossing Kingdoms: Using Decellularized Plants as Perfusable Tissue Engineering Scaffolds,” Biomaterials, 2017, Vol. 125, Pages 13-22; and R. J. Hickey, et al., “Cellulose Biomaterials for Tissue Engineering,” Frontiers in Bioengineering and Biotechnology, 2019, Vol. 7, 45.

The present invention also contemplates assaying the scaffolding and comparing such to collagen microcarriers in terms of cell growth. It is hypothesized that the scaffolding described herein will perform better for myotube differentiation than unorganized differentiation in plates because it allows a 3D structure.

Assays that may be used to assess the amount and effectiveness of the decellurization process include assaying DNA levels when measuring by PicoGreen and visually assessing the absence or presence of cell nuclei. Collagen and other ECM components can be assayed and quantified by hydroxyproline content, by sGAG, by histological analysis, and by SDS-PAGE. An additional assay that may be performed includes gelation kinetics assessed by turbidimetric analysis. Other assays include the use of antibodies to look for immunogenic antigens that may or may not be present on the decellurized seaweed.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method to decellularize an algae to form a scaffold for cell seeding, the method comprising:
chopping and grinding the algae;
adding the algae and a first solution to a tube at a first temperature for a first time period;
centrifuging the tube for a second time period and removing a supernatant;
washing with distilled water;
adding a second solution to the tube and incubating the sample at a second temperature for a third time period;
adding a third solution to the tube and incubating at a third temperature for a fourth time period;
adding a fourth solution to the tube and incubating at a fourth temperature for a fifth time period;
centrifuging the tube for a sixth time period and removing a supernatant;
adding distilled water to the tube and storing the algae and the distilled water at a fifth temperature until use.

2. The method of claim 1, wherein the algae is a species of brown algae.

3. The method of claim 2, wherein the algae is Durvillaea antarctica.

4. The method of claim 1, wherein each of the first temperature and the third temperature are room temperature.

5. The method of claim 1, wherein the second temperature is 60° C.

6. The method of claim 1, wherein the fourth temperature is around a boiling point of the fourth solution.

7. The method of claim 1, wherein the fifth temperature is about 4° C.

8. The method of claim 1, wherein the first time period is about three days.

9. The method of claim 1, wherein each of the second and sixth time period are about five minutes.

10. The method of claim 1, wherein the third time period is about two hours.

11. The method of claim 1, wherein the fourth time period is about five hours.

12. The method of claim 1, wherein the fifth time period is about 10 to 15 minutes.

13. The method of claim 1, wherein the first solution comprises an SDS solution.

14. The method of claim 1, wherein the second solution comprises a sodium acetate, acetic acid, and sodium chlorite solution.

15. The method of claim 1, wherein the third solution comprises a sodium hydroxide solution.

16. The method of claim 1, wherein the fourth solution comprises a hydrochloric acid solution.

17. The method of claim 1, wherein the method further comprises:
thawing the algae in sterilized water at a sixth temperature for a seventh time period for five times.

18. The method of claim 17, wherein the sixth temperature is about room temperature, and wherein the seventh time period is in a range of about 5 minutes to about 10 minutes.

19. The method of claim 17, further comprising:
centrifuging the sample for an eighth time period and extracting the supernatant.

20. The method of claim 19, wherein the eighth time period is about five minutes.

21. The method of claim 19, further comprising:
thawing the sample in a fifth solution at a seventh temperature for a ninth time period.

22. The method of claim 21, wherein the fifth solution comprises a sterilized PBS solution.

23. The method of claim 21, wherein the seventh temperature is room temperature, and wherein the ninth time period is about one hour.

24. The method of claim 21, further comprising:
transferring the algae, in a laminar flow hood, to tubes, adding a gelatin or albumin or RGD peptides and a sixth solution to generate a sample, allowing the sample to stand for a tenth time period, and storing the sample at an eighth temperature for an eleventh time period.

25. The method of claim 24, wherein the sixth solution comprises a sterilized CaCl₂) solution.

26. The method of claim 24, wherein the eighth temperature is about 37° C., and wherein the tenth time period is about one hour, and the eleventh time period is about twenty-four hours.

27. The method of 24, further comprising:
centrifuging the sample for an twelfth time period and removing the supernatant in the laminar flow hood such that the scaffold is ready for cell seeding.

28. The method of claim 27, wherein the twelfth time period is about five minutes.

29. The method of claim 27, wherein the scaffold is composed of sterile decellularized cochayuyo rehydrated with adhesion/carrier proteins.

30. The method of claim 27, wherein each of the adhesion/carrier proteins comprise one or more of 0.1-5% w/v gelatin, 0.1-5% % w/v albumin, or RGD peptides, and optionally another molecule.

31. The method of claim 27, wherein the scaffold is edible.

32. The method of claim 27, further comprising:
assaying the scaffold and comparing the scaffold to collagen microcarriers in terms of cell growth.
